(19) United States
(12) Patent Application Publication
(10) Pub. No.: US 2025/0214982 A1
(43) Pub. Date: Jul. 3, 2025

(54) COMPOUNDS USABLE AS MODULATORS OF PERK ACTIVITY

(71) Applicant: Ramot at Tel-Aviv University Ltd., Tel-Aviv (IL)

(72) Inventors: Gerardo Zelmar LEDERKREMER, Tel Aviv (IL); Marcelo EHRLICH, Tel Aviv (IL); Marina SHENKMAN, Tel Aviv (IL); Elvira HAIMOV, Tel Aviv (IL)

(73) Assignee: Ramot at Tel-Aviv University Ltd., Tel-Aviv (IL)

(21) Appl. No.: 18/851,760

(22) PCT Filed: Mar. 29, 2023
(86) PCT No.: PCT/IL2023/050329

§ 371 Date: Sep. 27, 2024

Related U.S. Application Data
(60) Provisional application No. 63/324,977, filed on Mar. 29, 2022.

(51) Int. Cl. C07D 413/14; A61K 31/505; A61K 31/5377; A61P 31/14; C07D 239/42

(52) U.S. Cl. CPC C07D 413/14; A61K 31/505; A61K 31/5377; A61P 31/14; C07D 239/42

(57) ABSTRACT

Newly designed compounds represented by Formulae I-V as described in the instant specification and uses thereof as pancreatic endoplasmic reticulum kinase (PERK) activators and in treating associated medical conditions, including viral infections, are provided.

### RELATED APPLICATION/S

This application claims the benefit of priority of U.S. Patent Application No. 63/324,977 filed on Mar. 29, 2022, the contents of which are incorporated herein by reference in their entirety.

### FIELD AND BACKGROUND OF THE INVENTION

The present invention, in some embodiments thereof, relates to therapy and, more particularly, but not exclusively, to newly designed substituted hydrazone and hydrazine compounds which are usable as PERK activators and in treating medical conditions associated with aggregation-prone protein and/or viral infections.

Proteins fold into their native conformation and undergo a series of post-translational modifications in the endoplasmic reticulum (ER) as part of the normal process of cellular homeostasis. Disruption of cellular protein folding results in ER stress. Cells respond to ER stress by activation of the unfolded protein response (UPR) pathways to survive the stress.

PERK, one of the three identified UPR transducers, is a kinase that phosphorylates a single known substrate eIF2α, leading to lower levels of translation initiation, which in turn globally reduces the load of newly synthesized proteins in the ER. Reduction in the overall protein-folding load is an effective response to reduce ER stress. In addition, PERK-mediated eIF2α phosphorylation also induces transcriptional activation to improve protein-folding capacity, thereby further promoting cell survival in stressed cells. Among the group of three prominent UPR transducers that includes also XBP1 and ATF6, PERK may have a broader range of cellular effects than other transducers, perhaps because of its unique role in regulating the general translation rate through the phosphorylation of eIF2α. Indeed, eIF2α phosphorylation appears to account for the entire range of the protective effects of PERK under ER stress. See, for example, Wang et al. [Chem Biol Drug Des 2010, 76:480-495], and references cited therein.

Small molecules modulators of PERK, such as GSK2606414, GSK2656157 and A4, have been designed primarily as cancer-treating drugs, but also as candidates for treating neurodegenerative diseases [see for example, Wang et al. (2010) supra; Axten et al. (2012) J Med Chem, 55:7193-7207; Axten et al. (2013) ACS Med Chem Lett, 4:964-968; Moreno et al. (2013) Sci Transl Med, 5 (206): 206ra138; Radford et al. (2015) Acta Neuropathol, 130:633-642; and International Patent Application Publications WO 2011/119663 and WO 2011/146748].

Hydrazone derivatives such as A4 and MK-28 have been found to modulate protein kinase R (PKR)-like endoplasmic reticulum kinase (PERK) [see, for example, Ganz et al. (2020) Scientific Reports, 10, 6875; and U.S. Pat. No. 11,104,651].

Hydrazone derivatives and PERK activators have been demonstrated to be useful in treating Huntington's disease, as well as other conditions associated with protein aggregation [see, for example, WO 2017/216792 and Ganz et al. (2020) supra].

U.S. Pat. No. 9,512,066 describes compounds suitable for modulating huntingtin processing and useful for treating huntingtin-related disorders.

U.S. Patent Application Publication No. 2010/0130473 describes hydrazone and hydrazide compounds which are ATP-competitive inhibitors of mTOR.

Hydrazone-gallate derivatives were also demonstrated to inhibit DNA methyltransferase 3A with high specificity [Erdmann et al. (2016) Future Medicinal Chemistry, 8 (4)], which may be useful in suppressing methylation-induced cancer.

### SUMMARY OF THE INVENTION

According to an aspect of some embodiments of the invention, there is provided a compound represented by Formula II:

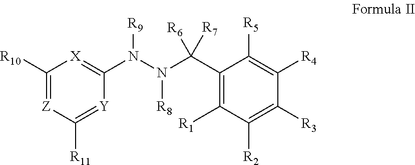

or a pharmaceutical acceptable salt thereof,
wherein:
X is N or CR₁₂;
Y is N or CR₁₃;
Z is N or CR₁₄;
R₁-R₇ and R₁₀-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carboxy, sulfonamido, guanyl, guanidinyl, hydrazine, hydrazide, thiohydrazide, and amino; and
R₈ and R₉ are each independently hydrogen or alkyl.

According to some of any of the embodiments described herein, X and Y are each N and Z is CR₁₄.

According to some of any of the embodiments described herein, R₁₀ and R₁₁ are each independently a substituted or non-substituted phenyl.

According to some of any of the embodiments described herein, at least one of R₈ and R₉ is alkyl.

According to some of any of the embodiments described herein, R₉ is methyl.

According to some of any of the embodiments described herein, R₆-R₈ are each hydrogen.

According to some of any of the embodiments described herein, at least one of R₂, R₃ and R₄ is hydroxy.

According to some of any of the embodiments described herein, at least two of R₂, R₃ and R₄ are hydroxy.

According to some of any of the embodiments described herein, R₂ and R₃ are each hydroxy.

According to some of any of the embodiments described herein, R₁, R₄ and R₅ are each hydrogen.

According to an aspect of some embodiments of the invention, there is provided a compound represented by Formula I:

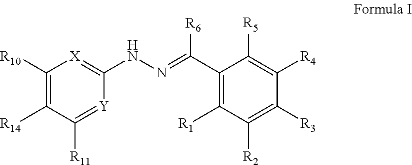

or a pharmaceutical acceptable salt thereof,
wherein:
X is N or CR₁₂;
Y is N or CR₁₃; and
R₁-R₆ and R₁₀-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

According to some of any of the embodiments described herein, at least one of X and Y is N.

According to some of any of the embodiments described herein, X and Y are each N.

According to some of any of the embodiments described herein, R₁₄ is hydrogen.

According to some of any of the embodiments described herein, at least two of R₂, R₃ and R₄ are each hydroxy.

According to some of any of the embodiments described herein, R₂, R₃ and R₄ are each hydroxy.

According to some of any of the embodiments described herein, R₁, R₅ and R₆ are each hydrogen.

According to some of any of the embodiments described herein, R₁₀ and R₁₁ are each independently a substituted or non-substituted phenyl.

According to some of any of the embodiments described herein, R₁₀ and R₁₁ are each hydrogen.

According to an aspect of some embodiments of the invention, there is provided a compound represented by Formula III:

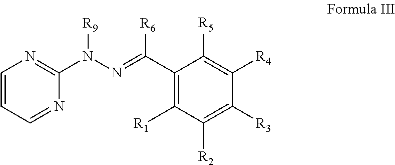

or a pharmaceutical acceptable salt thereof,
wherein:
R₁-R₆ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino; and R₉ is alkyl.

According to some of any of the embodiments described herein, at least two of R₁-R₅ are each independently hydroxy or alkoxy.

According to some of any of the embodiments described herein, at least two of R₂, R₃ and R₄ are each hydroxy.

According to some of any of the embodiments described herein, R₂, R₃ and R₄ are each hydroxy.

According to some of any of the embodiments described herein, R₁, R₅ and R₆ are each hydrogen.

According to some of any of the embodiments described herein, R₉ is methyl.

According to an aspect of some embodiments of the invention, there is provided a compound represented by Formula IV:

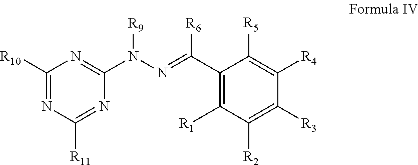

or a pharmaceutical acceptable salt thereof,
wherein:
R₁-R₆, R₁₀ and R₁₁ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carboxy, sulfonamido, guanyl, guanidinyl, hydrazine, hydrazide, thiohydrazide, and amino;
R₉ is hydrogen or alkyl; and
at least one, or each, of R₁₀ and R₁₁ is a heteroalicyclic.

According to some of any of the embodiments described herein, R₉ is hydrogen.

According to some of any of the embodiments described herein, R₁₀ and R₁₁ are each a heteroalicyclic.

According to some of any of the embodiments described herein, at least one of R₁-R₅ is a trihaloalkyl.

According to some of any of the embodiments described herein, at least one of R₂-R₄ is a trihaloalkyl.

According to some of any of the embodiments described herein, R₃ is a trihaloalkyl.

According to some of any of the embodiments described herein, at least one of R₁-R₅ is hydroxy.

According to some of any of the embodiments described herein, at least two of R₂, R₃ and R₄ are each hydroxy.

According to some of any of the embodiments described herein, R₂ and R₃ are each hydroxy, and R₁, R₄, R₅ and R₉ are each hydrogen.

According to some of any of the embodiments described herein, R₁ and R₄-R₆ are each hydrogen.

According to an aspect of some embodiments of the invention, there is provided a compound represented by Formula V:

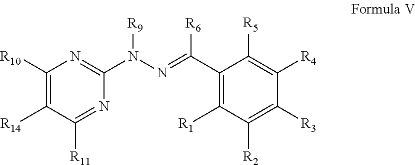

or a pharmaceutical acceptable salt thereof,
wherein:

R₁-R₆, R₁₀, R₁₁ and R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, with the proviso that neither R₂ nor R₄ is hydroxy or alkoxy;
at least one of R₁-R₅ is a trihaloalkyl;
R₉ is alkyl; and
at least one of R₁₀ and R₁₁ is other than hydrogen.

According to some of any of the embodiments described herein, R₁₀ and R₁₁ are each independently a substituted or non-substituted phenyl.

According to some of any of the embodiments described herein, at least one of R₂-R₄ is a trihaloalkyl.

According to some of any of the embodiments described herein, R₃ is a trihaloalkyl.

According to some of any of the embodiments described herein, none of R₁-R₅ is hydroxy or alkoxy.

According to some of any of the embodiments described herein, R₁, R₂ and R₄-R₆ are each hydrogen.

According to some of any of the embodiments described herein, R₉ is methyl.

According to some of any of the embodiments described herein, the compound is for use in treating a medical condition (e.g., a disease or disorder) in which activating PERK is beneficial.

According to some of any of the embodiments described herein, the medical condition is associated with an aggregation-prone protein.

According to some of any of the embodiments described herein, the medical condition associated with an aggregation-prone protein is selected from the group consisting of Huntington's disease, amyloidosis, cataract, type II diabetes, cancer and memory deficiency.

According to some of any of the embodiments described herein, the medical condition is a viral infection.

According to some of any of the embodiments described herein, the compound is for use in treating a medical condition in which upregulating an unfolded protein response is beneficial.

According to some of any of the embodiments described herein, the medical condition associated with an aggregation-prone protein is selected from the group consisting of Huntington's disease, amyloidosis, cataract, type II diabetes, cancer and memory deficiency.

According to some of any of the embodiments described herein, the compound is for use in treating a medical condition (e.g., a disease or disorder) associated with an aggregation-prone protein.

According to some of any of the embodiments described herein, the medical condition is selected from the group consisting of Huntington's disease, amyloidosis, cataract, type II diabetes, cancer and memory deficiency.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents comparative plots showing the apoptosis rate of MEF cells from UPR-induced apoptosis as a function of concentration (0-10 μM) of exemplary compounds according to some embodiments of the present invention, following 48 hours incubation of MEF cells with the ER stress-inducer tunicamycin. P values *<0.05, **<0.01, ***<0.001. The asterisks indicate the significance compared to untreated cells.

FIG. 2 presents comparative plots showing the apoptosis rate of STHdh^{Q111/111} cells as a function of concentration of the exemplary compounds GLB11 and GLC19, following incubation with tunicamycin for 48 hours), as described for FIG. 1. P values *<0.05, **<0.01, ***<0.001. The asterisks indicate the significance compared to untreated cells.

FIG. 3A presents comparative bar graphs showing the effect of treatment with MK-28 or GLB7 (5 μM, 10 μM or 20 μM, as indicated), on the levels of eIF2α-phosphorylation (eIF2α-P) relatively to total eIF2α (eIF2α) in lysed HEK293 cells. Treatment with MK-28 began 24 hours before lysis. Data was obtained from quantification of Western blots (FIG. 3B), and immunoblotting was performed with anti-eIF2α-P or anti-total eIF2α antibodies.

### DESCRIPTION OF SPECIFIC EMBODIMENTS OF THE INVENTION

The present invention, in some embodiments thereof, relates to therapy and, more particularly, but not exclusively, to newly designed substituted hydrazone and hydrazine compounds which are usable as PERK activators and in treating medical conditions associated with aggregation-prone protein and/or viral infections.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Following the design and successful practice of hydrazone compounds as modulators of PERK, as described, for example, in WO 2017/216792 and Ganz et al. (2020), and in a search for additional, preferably improved, PERK modulators, the present inventors have studied the effect of various structural modifications of these compounds on their activity as PERK modulators.

The present inventors have designed and successfully synthesized (see, Table 1 in the Examples section that follows) a set of exemplary hydrazone and hidrazine compounds and have tested the effect of these compounds as PERK modulators and on ER stress-induced apoptosis.

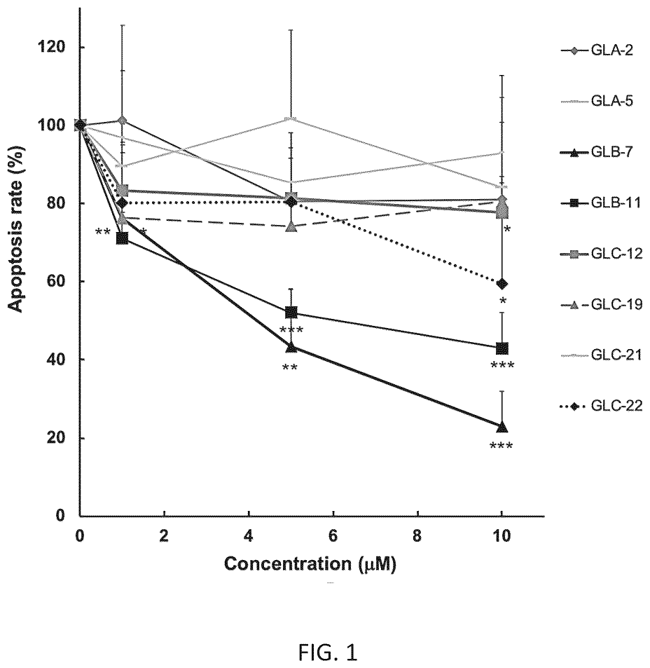

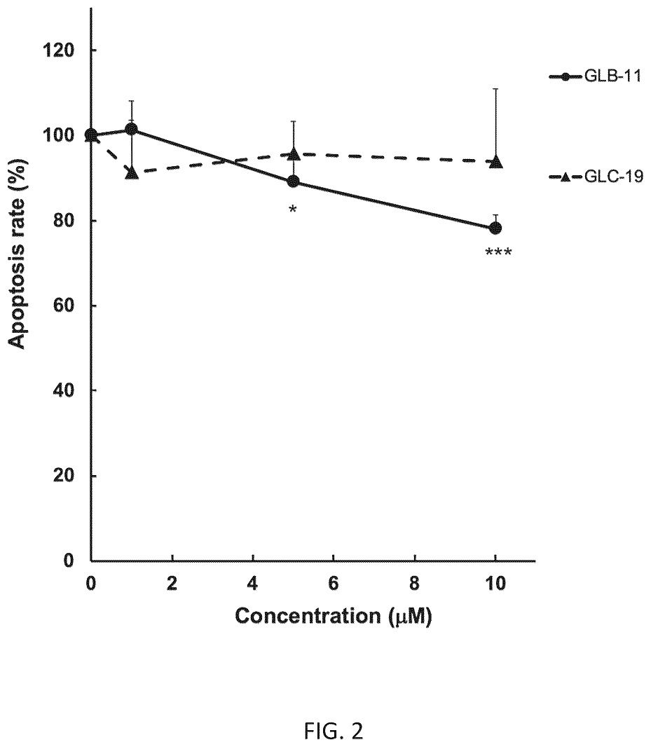

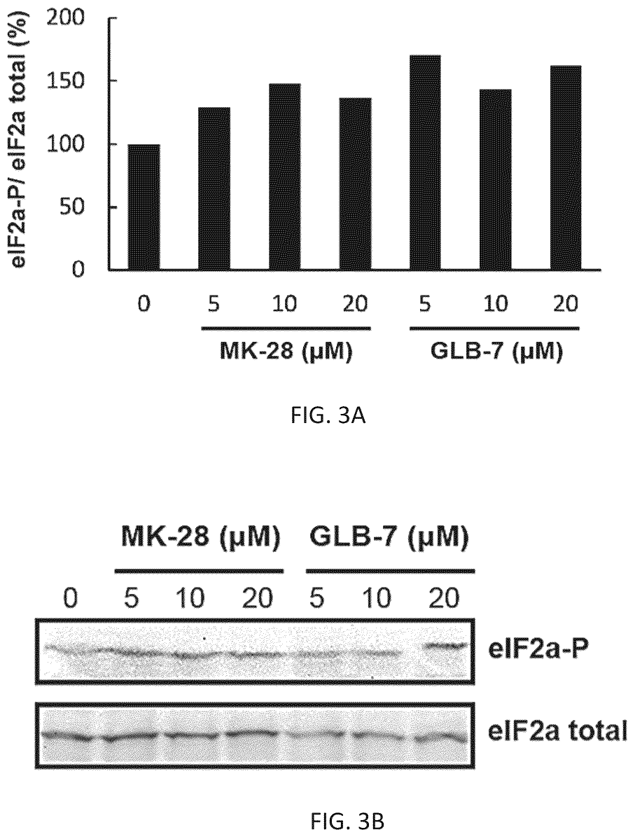

Table 2 in the Examples section that follows and FIGS. 1-3 present the data obtained in these studies, and demonstrate the dose-dependent effect of these compound in reducing apoptosis (FIGS. 1-2), and the effect of an exemplary compound on PERK activation (FIG. 3). Some of these compounds were further characterized in solubility and lipophilicity studies in order to assess their druggability, as present in Table 3 in the Examples section that follows.

The newly designed compounds are collectively represented herein by Formulae I-V, each representing a newly designed structural modification. Embodiments of the present invention relate to the compounds represented by Formulae I-V, and to uses thereof as pancreatic endoplasmic reticulum kinase (PERK) modulators (e.g., activators). Exemplary such compounds are shown in Table 2 in the Examples section that follows. Modulation of PERK can affect the cellular integrated stress response (ISR). Activation of PERK can upregulate an unfolded protein response, which is usable in treating diseases associated with aggregation-prone proteins, such as, but not limited to, Huntington's disease and other diseases, as described in further detail hereinunder. Activation of PERK can further boost the cellular integrated stress response (ISR) and reduce viral protein synthesis, rendering these compounds also usable in treating viral infections.

### Compounds:

According to an aspect of some embodiments of the present invention there is provided a compound represented by Formula I:

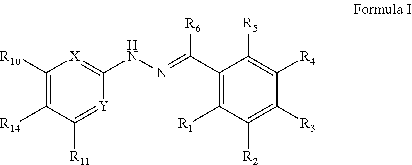

or a pharmaceutical acceptable salt thereof,
wherein:
X is N or CR₁₂;
Y is N or CR₁₃; and
R₁-R₆ and R₁₀-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

According to some of any of the embodiments of Formula I, at least one of X and Y is N.

According to some of any of the embodiments of Formula I, X and Y are each nitrogen (N).

According to some of any of the embodiments of Formula I, R₁₄ is hydrogen.

According to some of any of the embodiments of Formula I, at least one of R₂, R₃ and R₄ is hydroxy.

According to some of any of the embodiments of Formula I, at least two of R₂, R₃ and R₄ are each hydroxy.

According to some of any of the embodiments of Formula I, each of R₂, R₃ and R₄ is hydroxy.

According to some of any of the embodiments of Formula I, at least one, or each, of X and Y is nitrogen; and R₂, R₃ and R₄ are each hydroxy.

According to some of any of the embodiments of Formula I, at least one of R₁, R₅ and R₆ is hydrogen.

According to some of any of the embodiments of Formula I, at least two of R₁, R₅ and R₆ are each hydrogen.

According to some of any of the embodiments of Formula I, R₁, R₅ and R₆ are each hydrogen.

According to some of any of the embodiments of Formula I, at least two of R₂, R₃ and R₄ are each hydroxy; and at least one, or at least two, or each, of R₁, R₅ and R₆ is hydrogen.

According to some of any of the embodiments of Formula I, R₂, R₃ and R₄ are each hydroxy; and at least one, or at least two, or each, of R₁, R₅ and R₆ is hydrogen.

According to some of any of the embodiments of Formula I, at least two of R₂, R₃ and R₄ are each hydroxy; and R₁, R₅ and R₆ are each hydrogen.

According to some of any of the embodiments of Formula I, R₂, R₃ and R₄ are each hydroxy; and R₁, R₅ and R₆ are each hydrogen.

According to some of any of the embodiments of Formula I, at least one, or each, of X and Y is nitrogen; R₂, R₃ and R₄ are each hydroxy; and R₁, R₅ and R₆ are each hydrogen.

According to some of any of the embodiments of Formula I, each of X and Y is nitrogen; R₂, R₃ and R₄ are each hydroxy; and R₁, R₅ and R₆ are each hydrogen.

According to some of any of the embodiments of Formula I, at least one, or each, of R₁₀ and R₁₁ is independently a substituted or non-substituted phenyl.

According to some of any of the embodiments of Formula I, R₁₀ and R₁₁ are each independently a substituted or non-substituted phenyl.

According to some of any of the embodiments of Formula I, at least one, or each, of R₁₀ and R₁₁ is hydrogen.

According to some of any of the embodiments of Formula I, R₁₀ and R₁₁ are each hydrogen.

According to some of any of the embodiments of Formula I, at least two of R₂, R₃ and R₄ are each hydroxy; at least one, or at least two, or each, of R₁, R₅ and R₆ is hydrogen; and R₁₀ and R₁₁ are each independently a substituted or non-substituted phenyl.

According to some of any of the embodiments of Formula I, R₂, R₃ and R₄ are each hydroxy; at least one, or at least two, or each, of R₁, R₅ and R₆ is hydrogen; and R₁₀ and R₁₁ are each independently a substituted or non-substituted phenyl.

According to some of any of the embodiments of Formula I, at least two of R₂, R₃ and R₄ are each hydroxy; and R₁, R₅ and R₆ are each hydrogen; and R₁₀ and R₁₁ are each independently a substituted or non-substituted phenyl.

According to some of any of the embodiments of Formula I, at least one, or each, of X and Y is nitrogen; at least two of R₂, R₃ and R₄ are each hydroxy; at least one, or at least two, or each, of R₁, R₅ and R₆ is hydrogen; and R₁₀ and Ru are each independently a substituted or non-substituted phenyl.

According to some of any of the embodiments of Formula I, R₂, R₃ and R₄ are each hydroxy; R₁, R₅ and R₆ are each hydrogen; and R₁₀ and R₁₁ are each independently a substituted or non-substituted phenyl.

According to some of any of the embodiments described herein for R₁₀ and R₁₁ being each independently a substituted or non-substituted phenyl, when the phenyl is a substituted phenyl, it can be substituted by one or more substituents as described hereinbelow. According to some of these embodiments, R₁₀ and R₁₁ are each a non-substituted phenyl.

According to some of any of the embodiments described herein, for R₁₀ and R₁₁ in any of the relevant Formulae as described herein can alternatively be each independently a substituted or non-substituted aryl, as described herein.

According to some of any of the embodiments of Formula I, at least one, or each, of R₁₀ and R₁₁ is independently selected from the group of hydrogen, alkyl, alkenyl, alkynyl, cycloalkyl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

According to some of any of the embodiments of Formula I, at least two of R₂, R₃ and R₄ are each hydroxy; at least one, or at least two, or each, of R₁, R₅ and R₆ is hydrogen; and at least one, or each, of R₁₀ and R₁₁ is independently selected from the group of hydrogen, alkyl, alkenyl, alkynyl, cycloalkyl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

According to some of any of the embodiments of Formula I, R₂, R₃ and R₄ are each hydroxy; at least one, or at least two, or each, of R₁, R₅ and is hydrogen; and at least one, or each, of R₁₀ and R₁₁ is independently selected from the group of hydrogen, alkyl, alkenyl, alkynyl, cycloalkyl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

According to some of any of the embodiments of Formula I, at least two of R₂, R₃ and R₄ are each hydroxy; and R₁, R₅ and R₆ are each hydrogen; and at least one, or each, of R₁₀ and R₁₁ is independently selected from the group of hydrogen, alkyl, alkenyl, alkynyl, cycloalkyl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

According to some of any of the embodiments of Formula I, R₂, R₃ and R₄ are each hydroxy; R₁, R₅ and R₆ are each hydrogen; and at least one, or each, of R₁₀ and R₁₁ is independently selected from the group of hydrogen, alkyl, alkenyl, alkynyl, cycloalkyl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

According to some of any of the embodiments of Formula I, at least one, or each, of R₁₀ and R₁₁ is hydrogen.

According to some of any of the embodiments of Formula I, at least two of R₂, R₃ and R₄ are each hydroxy; at least one, or at least two, or each, of R₁, R₅ and R₆ is hydrogen; and at least one, or each, of R₁₀ and R₁₁ is hydrogen.

According to some of any of the embodiments of Formula I, R₂, R₃ and R₄ are each hydroxy; at least one, or at least two, or each, of R₁, R₅ and R₆ is hydrogen; and at least one, or each, of R₁₀ and R₁₁ is hydrogen.

According to some of any of the embodiments of Formula I, at least two of R₂, R₃ and R₄ are each hydroxy; and R₁, R₅ and R₆ are each hydrogen; and at least one, or each, of R₁₀ and R₁₁ is hydrogen.

According to some of any of the embodiments of Formula I, R₂, R₃ and R₄ are each hydroxy; R₁, R₅ and R₆ are each hydrogen; and at least one, or each, of R₁₀ and R₁₁ is hydrogen.

Exemplary compounds of Formula I include GLB7 and GLC21 (see, Table 2).

According to some of any of the embodiments described herein, the compound is 4-((2-(4,6-diphenylpyrimidin-2-yl)hydrazono)methyl)benzene-1,2-diol, which is also referred to herein as GLB7. According to some of any of the embodiments described herein, the compound is GLB7 or a pharmaceutical acceptable salt thereof.

In some of any of the embodiments described herein, “GLB7” as described herein encompasses, unless otherwise indicated, the (E)- and (Z)-4-((2-(4,6-diphenylpyrimidin-2-yl)hydrazono)methyl)benzene-1,2-diol:

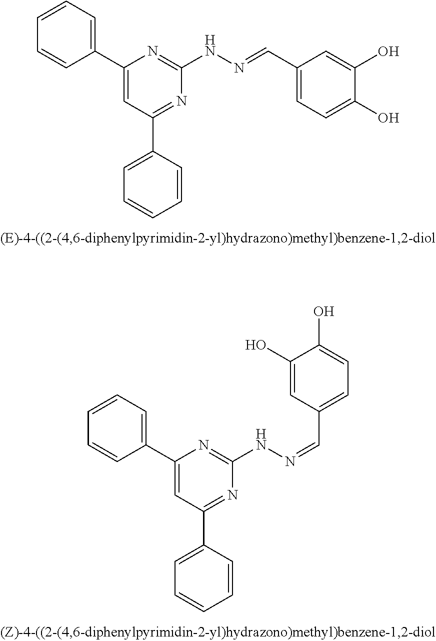 [SMILES: Oc1ccc(/C=N/Nc2nc(-c3ccccc3)cc(-c3ccccc3)n2)cc1O ; Oc1ccc(/C=N\Nc2nc(-c3ccccc3)cc(-c3ccccc3)n2)cc1O]

According to some of any of the embodiments described herein, the compound is (E)-5-((2-(pyrimidin-2-yl)hydrazono)methyl)benzene-1,2,3-triol, which is also referred to herein as GLC21, as shown below. According to some of any of the embodiments described herein, the compound is GLC21 or a pharmaceutical acceptable salt thereof.

In some of any of the embodiments described herein, the compound is (Z)-5-((2-(pyrimidin-2-yl)hydrazono)methyl)benzene-1,2,3-triol, as shown below.

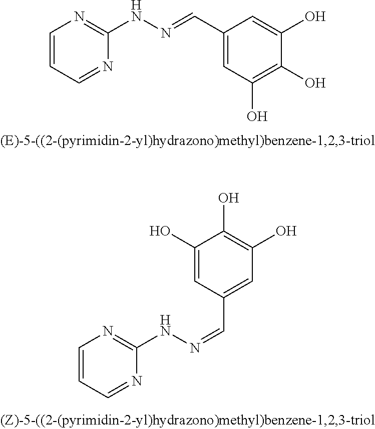 [SMILES: Oc1cc(/C=N/Nc2ncccn2)cc(O)c1O ; Oc1cc(/C=N\Nc2ncccn2)cc(O)c1O]

According to an aspect of some embodiments of the present invention there is provided a compound represented by Formula II:

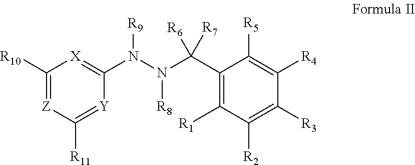

or a pharmaceutical acceptable salt thereof,
wherein:
X is N or CR₁₂;
Y is N or CR₁₃;
Z is N or CR₁₄;
R₁-R₇ and R₁₀-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino; and R₈ and R₉ are each independently hydrogen or alkyl.

According to some of any of the embodiments of Formula II as described herein, at least one of X, Y and Z is nitrogen (N).

According to some of any of the embodiments of Formula II as described herein, at least two of X, Y and Z are each nitrogen.

According to some of any of the embodiments of Formula II as described herein, at least one, or each, of X and Y is nitrogen.

According to some of any of the embodiments of Formula II as described herein, X and Y are each nitrogen.

According to some of any of the embodiments of Formula II as described herein, Z is CR₁₄. According to some of any of the embodiments described herein R₁₄ is hydrogen such that Z is CH.

According to some of any of the embodiments of Formula II as described herein, X and Y are each N and Z is CR₁₄, preferably Z is CH.

According to some of any of the embodiments of Formula II as described herein, R₁₀ and R₁₁ are each independently a substituted or non-substituted phenyl, as described herein.

According to some of any of the embodiments of Formula II as described herein, at least one, or each, of X and Y is nitrogen and Z is CR₁₄, preferably Z is CH; and R₁₀ and R₁₁ are each independently a substituted or non-substituted phenyl, as described herein.

According to some of any of the embodiments of Formula II as described herein, X and Y are each N and Z is CH; and R₁₀ and R₁₁ are each independently a substituted or non-substituted phenyl, as described herein.

According to some of any of the embodiments of Formula II as described herein, at least one, or each, of X and Y is nitrogen; and R₁₀ and R₁₁ are each independently a substituted or non-substituted phenyl, as described herein.

According to some of any of the embodiments of Formula II as described herein, at least one, or each, of R₈ and R₉ is alkyl.

According to some of any of the embodiments of Formula II as described herein, at least one, or each, of X and Y is nitrogen and Z is CR₁₄, preferably Z is CH; and at least one of R₈ and R₉ is alkyl.

According to some of any of the embodiments of Formula II as described herein, X and Y are each N and Z is CR₁₄, preferably Z is CH; and at least one of R₈ and R₉ is alkyl.

According to some of any of the embodiments of Formula II as described herein, at least one, or each, of X and Y is nitrogen; and at least one of R₈ and R₉ is alkyl.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; and at least one of R₈ and R₉ is alkyl.

According to some of any of the embodiments of Formula II as described herein, X and Y are each N and Z is CR₁₄, preferably Z is CH; R₁₀ and R₁₁ are each independently a substituted or non-substituted phenyl; and at least one of R₈ and R₉ is alkyl.

According to some of any of the embodiments of Formula II as described herein, at least one, or each, of X and Y are each nitrogen; R₁₀ and R₁₁ are each independently a substituted or non-substituted phenyl; and at least one of R₈ and R₉ is alkyl.

In some any of the embodiments described herein for Formula II, for R₈ and/or R₉ being an alkyl, the alkyl is a lower alkyl, of from 1 to 10, or from 1 to 8, or from 1 to 6, preferably from 1 to 4, carbon atoms in length. In some of these embodiments, the alkyl is a linear alkyl. In some any of the embodiments described herein for R₈ and/or R₉ being an alkyl, the alkyl can be substituted or unsubstituted, and is preferably unsubstituted.

In some any of the embodiments described herein for R₈ and/or R₉ being an alkyl, the alkyl is a linear, unsubstituted lower alkyl as described herein.

In exemplary embodiments, R₈ and/or R₉ is independently an alkyl selected from methyl, ethyl, propyl and butyl.

In exemplary embodiments, one of R₈ and R₉ is an alkyl as described herein in any of the respective embodiments, for example, a linear, unsubstituted lower alkyl as described herein, and the other one is hydrogen.

In some of any of the embodiments described herein, R₈ is hydrogen and R₉ is an alkyl as described herein in any of the respective embodiments, for example, a linear, unsubstituted lower alkyl as described herein.

According to some of any of the embodiments of Formula II as described herein, R₉ is a linear, unsubstituted lower alkyl as described herein, for example, methyl.

According to some of any of the embodiments of Formula II as described herein, at least one, or each, of X and Y is nitrogen and Z is CR₁₄, preferably Z is CH; and R₉ is a linear, unsubstituted lower alkyl as described herein, for example, methyl. In some of these embodiments, R₈ is hydrogen.

According to some of any of the embodiments of Formula II as described herein, X and Y are each N and Z is CR₁₄, preferably Z is CH; and R₉ is a linear, unsubstituted lower alkyl as described herein, for example, methyl. In some of these embodiments, R₈ is hydrogen.

According to some of any of the embodiments of Formula II as described herein, at least one, or each, of X and Y is nitrogen; and R₉ is a linear, unsubstituted lower alkyl as described herein, for example, methyl. In some of these embodiments, R₈ is hydrogen.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; and R₉ is a linear, unsubstituted lower alkyl as described herein, for example, methyl. In some of these embodiments, R₈ is hydrogen.

According to some of any of the embodiments of Formula II as described herein, X and Y are each N and Z is CR₁₄, preferably Z is CH; R₁₀ and R₁₁ are each independently a substituted or non-substituted phenyl; and R₉ is a linear, unsubstituted lower alkyl as described herein, for example, methyl. In some of these embodiments, R₈ is hydrogen.

According to some of any of the embodiments of Formula II as described herein, at least one, or each, of X and Y is nitrogen; R₁₀ and R₁₁ are each independently a substituted or non-substituted phenyl; and R₉ is a linear, unsubstituted lower alkyl as described herein, for example, methyl. In some of these embodiments, R₈ is hydrogen.

According to some of any of the embodiments of Formula II as described herein, R₆, R₇ and R₈ are each hydrogen.

According to some of any of the embodiments of Formula II as described herein, at least one, or each, of X and Y is nitrogen and Z is CH; and R₆, R₇ and R₈ are each hydrogen.

According to some of any of the embodiments of Formula II as described herein, X and Y are each N and Z is CR₁₄, preferably Z is CH; and R₆, R₇ and R₈ are each hydrogen.

According to some of any of the embodiments of Formula II as described herein, at least one, or each, of X and Y is nitrogen; and R₆, R₇ and R₈ are each hydrogen.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; and R₆, R₇ and R₈ are each hydrogen.

According to some of any of the embodiments of Formula II as described herein, X and Y are each N and Z is CR₁₄, preferably Z is CH; R₁₀ and R₁₁ are each independently a substituted or non-substituted phenyl; and R₆, R₇ and R₈ are each hydrogen.

According to some of any of the embodiments of Formula II as described herein, at least one, or each, of X and Y is nitrogen; R₁₀ and R₁₁ are each independently a substituted or non-substituted phenyl; and R₆, R₇ and R₈ are each hydrogen.

According to some of any of the embodiments of Formula II as described herein, at least one, or each, of X and Y is nitrogen and Z is CR₁₄, preferably Z is CH; R₆, R₇ and R₈ are each hydrogen are each hydrogen; and R₉ is a linear, unsubstituted lower alkyl as described herein, for example, methyl.

According to some of any of the embodiments of Formula II as described herein, X and Y are each N and Z is CR₁₄, preferably Z is CH; R₆, R₇ and R₈ are each hydrogen; and R₉ is a linear, unsubstituted lower alkyl as described herein, for example, methyl.

According to some of any of the embodiments of Formula II as described herein, at least one, or each, of X and Y is nitrogen; R₆, R₇ and R₈ are each hydrogen; and R₉ is a linear, unsubstituted lower alkyl as described herein, for example, methyl.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; R₆, R₇ and R₈ are each hydrogen; and R₉ is a linear, unsubstituted lower alkyl as described herein, for example, methyl.

According to some of any of the embodiments of Formula II as described herein, X and Y are each N and Z is CR₁₄, preferably Z is CH; R₁₀ and R₁₁ are each independently a substituted or non-substituted phenyl; R₆, R₇ and R₈ are each hydrogen; and R₉ is a linear, unsubstituted lower alkyl as described herein, for example, methyl.

According to some of any of the embodiments of Formula II as described herein, at least one, or each, of X and Y is nitrogen; R₁₀ and R₁₁ are each independently a substituted or non-substituted phenyl; R₆, R₇ and R₈ are each hydrogen; and R₉ is a linear, unsubstituted lower alkyl as described herein, for example, methyl.

According to some of any of the embodiments of Formula II as described herein, at least one of R₂, R₃ and R₄ is hydroxy.

According to some of any of the embodiments of Formula II as described herein, at least two, or each, of R₂, R₃ and R₄ is hydroxy.

According to some of any of the embodiments of Formula II as described herein, at least one, or each, of X and Y is nitrogen and Z is CR₁₄, preferably Z is CH; and at least two, or each, of R₂, R₃ and R₄ is hydroxy.

According to some of any of the embodiments of Formula II as described herein, X and Y are each N and Z is CH; and at least two, or each, of R₂, R₃ and R₄ is hydroxy.

According to some of any of the embodiments of Formula II as described herein, at least one, or each, of X and Y is nitrogen; and at least two, or each, of R₂, R₃ and R₄ is hydroxy.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; and at least two, or each, of R₂, R₃ and R₄ is hydroxy.

According to some of any of the embodiments of Formula II as described herein, X and Y are each N and Z is CR₁₄, preferably Z is CH; R₁₀ and R₁₁ are each independently a substituted or non-substituted phenyl; and at least two, or each, of R₂, R₃ and R₄ is hydroxy.

According to some of any of the embodiments of Formula II as described herein, at least one, or each, of X and Y is nitrogen; R₁₀ and R₁₁ are each independently a substituted or non-substituted phenyl; and at least two, or each, of R₂, R₃ and R₄ is hydroxy.

According to some of any of the embodiments of Formula II as described herein, R₆, R₇ and R₈ are each hydrogen; and at least two, or each, of R₂, R₃ and R₄ is hydroxy.

According to some of any of the embodiments of Formula II as described herein, at least one, or each, of X and Y is nitrogen and Z is CR₁₄, preferably Z is CH; R₆, R₇ and R₈ are each hydrogen; and at least two, or each, of R₂, R₃ and R₄ is hydroxy.

According to some of any of the embodiments of Formula II as described herein, X and Y are each N and Z is CR₁₄, preferably Z is CH; R₆, R₇ and R₈ are each hydrogen; and at least two, or each, of R₂, R₃ and R₄ is hydroxy.

According to some of any of the embodiments of Formula II as described herein, at least one, or each, of X and is nitrogen; R₆, R₇ and R₈ are each hydrogen; and at least two, or each, of R₂, R₃ and R₄ is hydroxy.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; R₆, R₇ and R₈ are each hydrogen; and at least two, or each, of R₂, R₃ and R₄ is hydroxy.

According to some of any of the embodiments of Formula II as described herein, X and Y are each N and Z is CR₁₄, preferably Z is CH; R₁₀ and R₁₁ are each independently a substituted or non-substituted phenyl; R₆, R₇ and R₈ are each hydrogen; and at least two, or each, of R₂, R₃ and R₄ is hydroxy.

According to some of any of the embodiments of Formula II as described herein, at least one, or each, of X and Y is nitrogen; R₁₀ and R₁₁ are each independently a substituted or non-substituted phenyl; R₆, R₇ and R₈ are each hydrogen; and at least two, or each, of R₂, R₃ and R₄ is hydroxy.

According to some of any of the embodiments of Formula II as described herein, at least one, or each, of X and Y is nitrogen and Z is CR₁₄, preferably Z is CH; R₆, R₇ and R₈ are each hydrogen; and at least two, or each, of R₂, R₃ and R₄ are hydroxy; and R₉ is a linear, unsubstituted lower alkyl as described herein, for example, methyl.

According to some of any of the embodiments of Formula II as described herein, X and Y are each N and Z is CR₁₄, preferably Z is CH; R₆, R₇ and R₈ are each hydrogen; at least two, or each, of R₂, R₃ and R₄ is hydroxy; and R₉ is a linear, unsubstituted lower alkyl as described herein, for example, methyl.

According to some of any of the embodiments of Formula II as described herein, at least one, or each, of X and Y is nitrogen; R₆, R₇ and R₈ are each hydrogen; at least two, or each, of R₂, R₃ and R₄ is hydroxy; and R₉ is a linear, unsubstituted lower alkyl as described herein, for example, methyl.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; R₆, R₇ and R₈ are each hydrogen; at least two, or each, of R₂, R₃ and R₄ is hydroxy; and R₉ is a linear, unsubstituted lower alkyl as described herein, for example, methyl.

According to some of any of the embodiments of Formula II as described herein, X and Y are each N and Z is CR₁₄, preferably Z is CH; R₁₀ and R₁₁ are each independently a substituted or non-substituted phenyl; R₆, R₇ and R₈ are each hydrogen; at least two, or each, of R₂, R₃ and R₄ is hydroxy; and R₉ is a linear, unsubstituted lower alkyl as described herein, for example, methyl.

According to some of any of the embodiments of Formula II as described herein, at least one, or each, of X and Y is nitrogen; R₁₀ and R₁₁ are each independently a substituted or non-substituted phenyl; R₆, R₇ and R₈ are each hydrogen; at least two, or each, of R₂, R₃ and R₄ is hydroxy; and R₉ is a linear, unsubstituted lower alkyl as described herein, for example, methyl. According to some of any of the embodiments of Formula II as described herein, R₂ and R₃ are each hydroxy. In some of any of these embodiments, R₄ is hydrogen.

According to some of any of the embodiments of Formula II as described herein, at least one, or each, of X and Y is nitrogen and Z is CR₁₄, preferably Z is CH; and R² and R₃ are each hydroxy. In some of any of these embodiments, R₄ is hydrogen.

According to some of any of the embodiments of Formula II as described herein, X and Y are each N and Z is CR₁₄, preferably Z is CH; and R² and R₃ are each hydroxy. In some of any of these embodiments, R₄ is hydrogen.

According to some of any of the embodiments of Formula II as described herein, at least one, or each, of X and Y is nitrogen; and R² and R₃ are each hydroxy. In some of any of these embodiments, R₄ is hydrogen.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; and R² and R₃ are each hydroxy. In some of any of these embodiments, R₄ is hydrogen.

According to some of any of the embodiments of Formula II as described herein, X and Y are each N and Z is CR₁₄, preferably Z is CH; R₁₀ and R₁₁ are each independently a substituted or non-substituted phenyl; and R² and R₃ are each hydroxy. In some of any of these embodiments, R₄ is hydrogen.

According to some of any of the embodiments of Formula II as described herein, at least one, or each, of X and Y is nitrogen; R₁₀ and R₁₁ are each independently a substituted or non-substituted phenyl; and R² and R₃ are each hydroxy. In some of any of these embodiments, R₄ is hydrogen.

According to some of any of the embodiments of Formula II as described herein, R₆, R₇ and R₈ are each hydrogen; and R² and R₃ are each hydroxy. In some of any of these embodiments, R₄ is hydrogen.

According to some of any of the embodiments of Formula II as described herein, at least one, or each, of X and Y is nitrogen and Z is CR₁₄, preferably Z is CH; R₆, R₇ and R₈ are each hydrogen; and R² and R₃ are each hydroxy. In some of any of these embodiments, R₄ is hydrogen.

According to some of any of the embodiments of Formula II as described herein, X and Y are each N and Z is CR₁₄, preferably Z is CH; R₆, R₇ and R₈ are each hydrogen; and R² and R₃ are each hydroxy. In some of any of these embodiments, R₄ is hydrogen.

According to some of any of the embodiments of Formula II as described herein, at least one, or each, of X and Y is nitrogen; R₆, R₇ and R₈ are each hydrogen; and R² and R₃ are each hydroxy. In some of any of these embodiments, R₄ is hydrogen.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; R₆, R₇ and R₈ are each hydrogen; and R² and R₃ are each hydroxy. In some of any of these embodiments, R₄ is hydrogen.

According to some of any of the embodiments of Formula II as described herein, X and Y are each N and Z is CR₁₄, preferably Z is CH; R₁₀ and R₁₁ are each independently a substituted or non-substituted phenyl; R₆, R₇ and R₈ are each hydrogen; and R² and R₃ are each hydroxy. In some of any of these embodiments, R₄ is hydrogen.

According to some of any of the embodiments of Formula II as described herein, at least one, or each, of X and Y is nitrogen; R₁₀ and R₁₁ are each independently a substituted or non-substituted phenyl; R₆, R₇ and R₈ are each hydrogen; and R² and R₃ are each hydroxy. In some of any of these embodiments, R₄ is hydrogen.

According to some of any of the embodiments of Formula II as described herein, at least one, or each, of X and Y is nitrogen and Z is CR₁₄, preferably Z is CH; R₆, R₇ and R₈ are each hydrogen; R₂ and R₃ are each hydroxy; and R₉ is a linear, unsubstituted lower alkyl as described herein, for example, methyl. In some of any of these embodiments, R₄ is hydrogen.

According to some of any of the embodiments of Formula II as described herein, X and Y are each N and Z is CR₁₄, preferably Z is CH; R₆, R₇ and R₈ are each hydrogen; R₂ and R₃ are each hydroxy; R₉ is a linear, unsubstituted lower alkyl as described herein, for example, methyl. In some of any of these embodiments, R₄ is hydrogen.

According to some of any of the embodiments of Formula II as described herein, at least one, or each, of X and Y is nitrogen; R₆, R₇ and R₈ are each hydrogen; R₂ and R₃ are each hydroxy; and R₉ is a linear, unsubstituted lower alkyl as described herein, for example, methyl. In some of any of these embodiments, R₄ is hydrogen.

According to some of any of the embodiments of Formula II as described herein, at least one, or each, of X and Y is nitrogen and Z is CR₁₄, preferably Z is CH; R₁₀ and Ru are each independently a substituted or non-substituted phenyl; R₆, R₇ and R₈ are each hydrogen; R₂ and R₃ are each hydroxy; and R₉ is a linear, unsubstituted lower alkyl as described herein, for example, methyl. In some of any of these embodiments, R₄ is hydrogen.

According to some of any of the embodiments of Formula II as described herein, X and Y are each N and Z is CR₁₄, preferably Z is CH; R₁₀ and R₁₁ are each independently a substituted or non-substituted phenyl; R₆, R₇ and R₈ are each hydrogen; R₂ and R₃ are each hydroxy; and R₉ is a linear, unsubstituted lower alkyl as described herein, for example, methyl. In some of any of these embodiments, R₄ is hydrogen.

According to some of any of the embodiments of Formula II as described herein, at least one, or each, of X and Y is nitrogen; R₁₀ and R₁₁ are each independently a substituted or non-substituted phenyl; R₆, R₇ and R₈ are each hydrogen; R₂ and R₃ are each hydroxy; and R₉ is a linear, unsubstituted lower alkyl as described herein, for example, methyl. In some of any of these embodiments, R₄ is hydrogen.

According to some of any of the embodiments of Formula II as described herein, at least one, at least two or each of R₁, R₄ and R₅ is hydrogen.

According to some of any of the embodiments of Formula II as described herein, each of R₁, R₄ and R₅ is hydrogen.

According to some of any of the embodiments of Formula II as described herein, at least one, or each, of X and Y is nitrogen and Z is CR₁₄, preferably Z is CH; and R₁, R₄ and R₅ are each hydrogen.

According to some of any of the embodiments of Formula II as described herein, X and Y are each N and Z is CR₁₄, preferably Z is CH; and R₁, R₄ and R₅ are each hydrogen.

According to some of any of the embodiments of Formula II as described herein, at least one, or each, of X and Y is nitrogen; and R₁, R₄ and R₅ are each hydrogen.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; and R₁, R₄ and R₅ are each hydrogen.

According to some of any of the embodiments of Formula II as described herein, X and Y are each N and Z is CR₁₄, preferably Z is CH; R₁₀ and R₁₁ are each independently a substituted or non-substituted phenyl; and R₁, R₄ and R₅ are each hydrogen.

According to some of any of the embodiments of Formula II as described herein, at least one, or each, of X and Y is nitrogen; R₁₀ and R₁₁ are each independently a substituted or non-substituted phenyl; and R₁, R₄ and R₅ are each hydrogen.

According to some of any of the embodiments of Formula II as described herein, at least one, or each, of X and is nitrogen and Z is CR₁₄, preferably Z is CH; at least two, or each, of R₂ and R₃ is hydroxy; and R₁, R₄ and R₅ are each hydrogen.

According to some of any of the embodiments of Formula II as described herein, X and Y are each N and Z is CR₁₄, preferably Z is CH; each of R₂ and R₃ is hydroxy; and R₁, R₄ and R₅ are each hydrogen.

According to some of any of the embodiments of Formula II as described herein, at least one, or each, of X and Y is nitrogen; each of R₂, and R₃ is hydroxy; and R₁, R₄ and R₅ are each hydrogen.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; each of R₂ and R₃ is hydroxy; and R₁, R₄ and R₅ are each hydrogen.

According to some of any of the embodiments of Formula II as described herein, X and Y are each N and Z is CR₁₄, preferably Z is CH; R₁₀ and R₁₁ are each independently a substituted or non-substituted phenyl; each of R₂ and R₃ is hydroxy; and R₁, R₄ and R₅ are each hydrogen.

According to some of any of the embodiments of Formula II as described herein, at least one, or each, of X and Y is nitrogen; R₁₀ and R₁₁ are each independently a substituted or non-substituted phenyl; R₂ and R₃ are each hydroxy; and R₁, R₄ and R₅ are each hydrogen.

According to some of any of the embodiments of Formula II as described herein, R₆-R₈ are each hydrogen; R₂ and R₃ are each hydroxy; and R₁, R₄ and R₅ are each hydrogen.

According to some of any of the embodiments of Formula II as described herein, at least one, or each, of X and Y is nitrogen and Z is CR₁₄, preferably Z is CH; R₆, R₇ and R₈ are each hydrogen; R₂ and R₃ are each hydroxy; and R₁, R₄ and R₅ are each hydrogen.

According to some of any of the embodiments of Formula II as described herein, X and Y are each N and Z is CR₁₄, preferably Z is CH; R₆, R₇ and R₈ are each hydrogen; R₂ and R₃ are each hydroxy; and R₁, R₄ and R₅ are each hydrogen.

According to some of any of the embodiments of Formula II as described herein, at least one, or each, of X and Y is nitrogen; R₆, R₇ and R₈ are each hydrogen; each of R₂ and R₃ is hydroxy; and R₁, R₄ and R₅ are each hydrogen.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; R₆, R₇ and R₈ are each hydrogen; each of R₂ and R₃ is hydroxy; and R₁, R₄ and R₅ are each hydrogen.

According to some of any of the embodiments of Formula II as described herein, X and Y are each N and Z is CR₁₄, preferably Z is CH; R₁₀ and R₁₁ are each independently a substituted or non-substituted phenyl; R₆, R₇ and R₈ are each hydrogen; each of R₂ and R₃ is hydroxy; and R₁, R₄ and R₅ are each hydrogen.

According to some of any of the embodiments of Formula II as described herein, at least one, or each, of X and Y is nitrogen; R₁₀ and R₁₁ are each independently a substituted or non-substituted phenyl; R₆, R₇ and R₈ are each hydrogen; each of R₂ and R₃ is hydroxy; and R₁, R₄ and R₅ are each hydrogen.

According to some of any of the embodiments of Formula II as described herein, at least one, or each, of X and Y is nitrogen and Z is CR₁₄, preferably Z is CH; R₆, R₇ and R₈ are each hydrogen; each of R₂ and R₃ is hydroxy; R₁, R₄ and R₅ are each hydrogen; and R₉ is a linear, unsubstituted lower alkyl as described herein, for example, methyl.

According to some of any of the embodiments of Formula II as described herein, X and Y are each N and Z is CR₁₄, preferably Z is CH; R₆, R₇ and R₈ are each hydrogen; each of R₂ and R₃ is hydroxy; R₁, R₄ and R₅ are each hydrogen; and R₉ is a linear, unsubstituted lower alkyl as described herein, for example, methyl.

According to some of any of the embodiments of Formula II as described herein, at least one, or each, of X and Y is nitrogen; R₆, R₇ and R₈ are each hydrogen; each of R₂ and R₃ is hydroxy; R₁, R₄ and R₅ are each hydrogen; and R₉ is a linear, unsubstituted lower alkyl as described herein, for example, methyl.

According to some of any of the embodiments of Formula II as described herein, at least one, or each, of X and Y is nitrogen and Z is CR₁₄, preferably Z is CH; R₁₀ and R₁₁ are each independently a substituted or non-substituted phenyl; R₆, R₇ and R₈ are each hydrogen; each of R₂ and R₃ is hydroxy; R₁, R₄ and R₅ are each hydrogen; and R₉ is a linear, unsubstituted lower alkyl as described herein, for example, methyl.

According to some of any of the embodiments of Formula II as described herein, X and Y are each N and Z is CR₁₄, preferably Z is CH; R₁₀ and R₁₁ are each independently a substituted or non-substituted phenyl; R₆, R₇ and R₈ are each hydrogen; each of R₂ and R₃ is hydroxy; R₁, R₄ and R₅ are each hydrogen; and R₉ is a linear, unsubstituted lower alkyl as described herein, for example, methyl.

According to some of any of the embodiments of Formula II as described herein, at least one, or each, of X and Y is nitrogen; R₁₀ and R₁₁ are each independently a substituted or non-substituted phenyl; R₆, R₇ and R₈ are each hydrogen; each of R₂ and R₃ is hydroxy; R₁, R₄ and R₅ are each hydrogen; and R₉ is a linear, unsubstituted lower alkyl as described herein, for example, methyl.

According to some of any of the embodiments described herein, the compound is 4-((2-(4,6-diphenylpyrimidin-2-yl)-2-methylhydrazinyl)methyl)benzene-1,2-diol), which is also referred to herein as GLB11 (see, Table 2). According to some of any of the embodiments described herein, the compound is GLB11 or a pharmaceutical acceptable salt thereof.

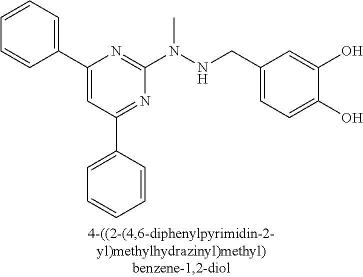 [SMILES: CN(NCc1ccc(O)c(O)c1)c1nc(-c2ccccc2)cc(-c2ccccc2)n1]

According to an aspect of some embodiments of the present invention there is provided a compound represented by Formula III:

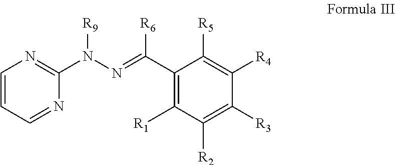

or a pharmaceutical acceptable salt thereof,
wherein:
R₁-R₆ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino; and
R₉ is alkyl.

According to some of any of the embodiments of Formula III as described herein, at least one, or at least two, or at least three, or each, of R₁-R₅ is independently hydroxy or alkoxy.

According to some of these embodiments, the alkoxy is a lower alkoxy, being of from 1 to 10, or from 1 to 8, or from 1 to 6, or from 1 to 4, carbon atoms in length. In some embodiments, the alkoxy is a linear alkoxy. In some embodiments, the alkoxy is a lower linear alkoxy. In some embodiments, the alkoxy is unsubstituted.

According to some of any of the embodiments of Formula III as described herein, at least two of R₁-R₅ are each independently hydroxy or an alkoxy as described herein.

According to some of any of the embodiments of Formula III as described herein, two of R₂, R₃ and R₄ are each hydroxy.

According to some of any of the embodiments of Formula III as described herein, R₂, R₃ and R₄ are each hydroxy.

According to some of any of the embodiments of Formula III as described herein, R₁, R₅ and R₆ are each hydrogen.

According to some of any of the embodiments of Formula III as described herein, R₃ and R₄ are each hydroxy and R₂ is hydrogen.

According to some of any of the embodiments of Formula III as described herein, R₃ and R₄ are each hydroxy, and R₁, R₂, R₅ and R₆ are each hydrogen

According to some of any of the embodiments of Formula III as described herein, two of R₂, R₃ and R₄ are each hydroxy; and R₁ and R₅ are each hydrogen. According to some of these embodiments, R₃ and R₄ are each hydroxy and R₂ is hydrogen.

According to some of any of the embodiments of Formula III as described herein, R₂, R₃ and R₄ are each hydroxy; and R₁ and R₅ are each hydrogen.

According to some of any of the embodiments of Formula III as described herein, two of R₂, R₃ and R₄ are each hydroxy; and R₁, R₅ and R₆ are each hydrogen. According to some of these embodiments, R₃ and R₄ are each hydroxy and R₂ is hydrogen.

According to some of any of the embodiments of Formula III as described herein, R₂, R₃ and R₄ are each hydroxy; and R₁, R₅ and R₆ are each hydrogen.

According to some of any of the embodiments of Formula III as described herein, R₉ is a linear, unsubstituted lower alkyl as described herein, for example, methyl.

According to some of any of the embodiments of Formula III as described herein, two of R₂, R₃ and R₄ are each hydroxy; and R₉ is alkyl, for example, R₉ is a linear, unsubstituted lower alkyl as described herein, for example, methyl. According to some of these embodiments, R₃ and R₄ are each hydroxy and R₂ is hydrogen.

According to some of any of the embodiments of Formula III as described herein, R₂, R₃ and R₄ are each hydroxy; and R₉ is alkyl, for example, R₉ is a linear, unsubstituted lower alkyl as described herein, for example, methyl.

According to some of any of the embodiments of Formula III as described herein, R₁, R₅ and R₆ are each hydrogen; and R₉ is alkyl, for example, R₉ is a linear, unsubstituted lower alkyl as described herein, for example, methyl.

According to some of any of the embodiments of Formula III as described herein, two of R₂, R₃ and R₄ are each hydroxy; R₁ and R₅ are each hydrogen; and R₉ is alkyl, for example, R₉ is a linear, unsubstituted lower alkyl as described herein, for example, methyl. According to some of these embodiments, R₃ and R₄ are each hydroxy and R₂ is hydrogen.

According to some of any of the embodiments of Formula III as described herein, R₂, R₃ and R₄ are each hydroxy; R₁ and R₅ are each hydrogen; and R₉ is alkyl, R₉ is a linear, unsubstituted lower alkyl as described herein, for example, methyl.

According to some of any of the embodiments of Formula III as described herein, two of R₂, R₃ and R₄ are each hydroxy; R₁, R₅ and R₆ are each hydrogen; and R₉ is alkyl, R₉ is a linear, unsubstituted lower alkyl as described herein, for example, methyl. According to some of these embodiments, R₃ and R₄ are each hydroxy and R₂ is hydrogen.

According to some of any of the embodiments of Formula III as described herein, R₂, R₃ and R₄ are each hydroxy; R₁, R₅ and R₆ are each hydrogen; and R₉ is alkyl, R₉ is a linear, unsubstituted lower alkyl as described herein, for example, methyl.

According to some of any of the embodiments of Formula III as described herein, two of R₂, R₃ and R₄ are each hydroxy; and R₉ is methyl. According to some of these embodiments, R₃ and R₄ are each hydroxy and R₂ is hydrogen.

According to some of any of the embodiments of Formula III as described herein, R₂, R₃ and R₄ are each hydroxy; and R₉ is methyl.

According to some of any of the embodiments of Formula III as described herein, R₁, R₅ and R₆ are each hydrogen; and R₉ is methyl.

According to some of any of the embodiments of Formula III as described herein, two of R₂, R₃ and R₄ are each hydroxy; R₁ and R₅ are each hydrogen; and R₉ is methyl. According to some of these embodiments, R₃ and R₄ are each hydroxy and R₂ is hydrogen.

According to some of any of the embodiments of Formula III as described herein, R₂, R₃ and R₄ are each hydroxy; R₁ and R₅ are each hydrogen; and R₉ is methyl.

According to some of any of the embodiments of Formula III as described herein, two of R₂, R₃ and R₄ are each hydroxy; R₁, R₅ and R₆ are each hydrogen; and R₉ is methyl. According to some of these embodiments, R₃ and R₄ are each hydroxy and R₂ is hydrogen.

According to some of any of the embodiments of Formula III as described herein, R₂, R₃ and R₄ are each hydroxy; R₁, R₅ and R₆ are each hydrogen; and R₉ is methyl.

According to some of any of the embodiments described herein, the compound is (E)-4-((2-methyl-2-(pyrimidin-2-yl)hydrazono)methyl)benzene-1,2-diol), which is also referred to herein as GLA2 as shown below (See, Table 2). According to some of any of the embodiments described herein, the compound is GLA2 or a pharmaceutical acceptable salt thereof.

In some of any of the embodiments described herein, the compound is (Z)-4-((2-methyl-2-(pyrimidin-2-yl)hydrazono)methyl)benzene-1,2-diol, as shown below.

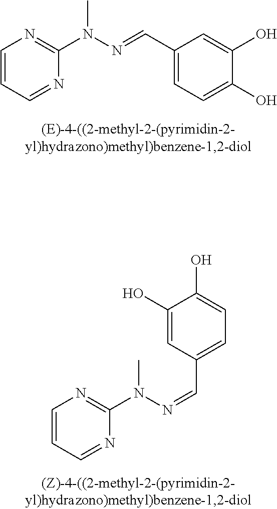 [SMILES: CN(/N=C/c1ccc(O)c(O)c1)c1ncccn1 ; CN(/N=C\c1ccc(O)c(O)c1)c1ncccn1]

According to some of any of the embodiments described herein, the compound is (E)-5-((2-methyl-2-(pyrimidin-2-yl)hydrazono)methyl)benzene-1,2,3-triol), which is also referred to herein as GLC22 as shown below (see also Table 2). According to some of any of the embodiments described herein, the compound is GLC22 or a pharmaceutical acceptable salt thereof.

In some of any of the embodiments described herein, the compound is (Z)-5-((2-methyl-2-(pyrimidin-2-yl)hydrazono)methyl)benzene-1,2,3-triol, as shown below.

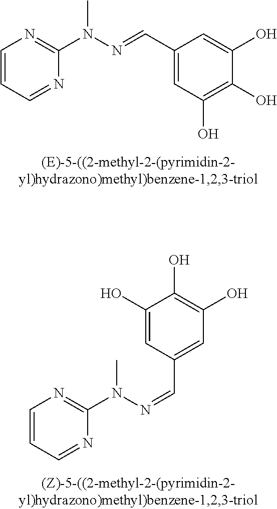 [SMILES: CN(/N=C/c1cc(O)c(O)c(O)c1)c1ncccn1 ; CN(/N=C\c1cc(O)c(O)c(O)c1)c1ncccn1]

According to an aspect of some embodiments of the present invention there is provided a compound represented by Formula IV:

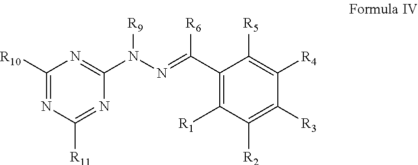

or a pharmaceutical acceptable salt thereof,
wherein:
R₁-R₆, R₁₀ and R₁₁ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carboxy, sulfonamido, guanyl, guanidinyl, hydrazine, hydrazide, thiohydrazide, and amino, at least one, or each, of R₁₀ and R₁₁ being a heteroalicyclic, as defined herein; and
R₉ is hydrogen or alkyl, preferably a lower alkyl as described herein for Formula I.

According to some of any of the embodiments of Formula IV as described herein, R₉ is hydrogen.

According to some of any of the embodiments of Formula IV as described herein, R₆ is hydrogen.

According to some of any of the embodiments of Formula IV as described herein, R₆ and R₉ are each hydrogen.

According to some of any of the embodiments of Formula IV as described herein, R₁₀ and R₁₁ are each independently a heteroalicyclic, which can be the same or different.

According to some of any of the embodiments described herein, the heteroalicylic is a nitrogen-containing heteroalicyclic, for example, piperidine, piperazine. Alternatively, the heteroalicyclic is an oxygen-containing heteroalicyclic, such as, for example, tetrahydrofuran, tetrahydropyran, dioxane. According to some of any of the embodiments described herein, the heteroalicylic contains two heteroatoms, for example, oxygen and nitrogen, for example, morpholine.

According to some of any of the embodiments of Formula IV as described herein, R₁₀ and R₁₁ are each the heteroalicyclic.

According to some of any of the embodiments of Formula IV as described herein, R₁₀ and R₁₁ are each morpholine.

According to some of any of the embodiments of Formula IV as described herein, R₉ is hydrogen; and R₁₀ and R₁₁ are each morpholine. In some of these embodiments, R₆ is hydrogen.

According to some of any of the embodiments of Formula IV as described herein, at least one of R₁-R₅ is a trihaloalkyl. In some of these embodiments, R₆ is hydrogen. In some of these embodiments, R₉ is hydrogen.

According to some of any of the embodiments of Formula IV as described herein, the trihaloalkyl is a lower trihaloalkyl, of from 1 to 10, or from 1 to 8, or from 1 to 6, preferably of from 1 to 4, carbon atoms in length. The three halo atoms can be the same or different. In some embodiments the three halo atoms are the same and is some embodiments, the trihaloalkyl is a trifluoroalkyl, preferably a trifluoroalkyl of 1 to 4 carbon atoms in length. In exemplary embodiments, the trihaloalkyl is trifluoromethyl.

According to some of any of the embodiments of Formula IV as described herein, R₉ is hydrogen; and at least one of R₁-R₅ is a trihaloalkyl, as described herein. In some of these embodiments, R₆ is hydrogen.

According to some of any of the embodiments of Formula IV as described herein, R₁₀ and R₁₁ are each a heteroalicyclic (e.g., are each morpholine); and at least one of R₁-R₅ is a trihaloalkyl, as described herein. In some of these embodiments, R₆ is hydrogen.

According to some of any of the embodiments of Formula IV as described herein, R₉ is hydrogen; R₁₀ and R₁₁ are each a heteroalicyclic (e.g., are each morpholine); and at least one of R₁-R₅ is a trihaloalkyl, as described herein. In some of these embodiments, R₆ is hydrogen.

According to some of any of the embodiments of Formula IV as described herein, at least one of R₂, R₃ and R₄ is a trihalomethyl, for example trifluoromethyl. In some of these embodiments, R₁ and R₅ are each hydrogen. In some of these embodiments, R₆ is hydrogen.

According to some of any of the embodiments of Formula IV as described herein, R₁₀ and R₁₁ are each a heteroalicyclic (e.g., are each morpholine); and at least one of R₂, R₃ and R₄ is trihaloalkyl, as described herein. In some of these embodiments, R₁ and R₅ are each hydrogen. In some of these embodiments, R₆ is hydrogen.

According to some of any of the embodiments of Formula IV as described herein, R₉ is hydrogen; and at least one of R₂, R₃ and R₄ is trihaloalkyl, as described herein. In some of these embodiments, R₁ and R₅ are each hydrogen. In some of these embodiments, R₆ is hydrogen.

According to some of any of the embodiments of Formula IV as described herein, R₁₀ and R₁₁ are each a heteroalicyclic (e.g., are each morpholine); and at least one of R₂, R₃ and R₄ is trihaloalkyl, as described herein. In some of these embodiments, R₁ and R₅ are each hydrogen. In some of these embodiments, R₆ is hydrogen.

According to some of any of the embodiments of Formula IV as described herein, R₉ is hydrogen; R₁₀ and R₁₁ are each a heteroalicyclic (e.g., are each morpholine); and at least one of R₂, R₃ and R₄ is trihaloalkyl, as described herein. In some of these embodiments, R₁ and R₅ are each hydrogen. In some of these embodiments, R₆ is hydrogen.

According to some of any of the embodiments of Formula IV as described herein, R₃ is a trihaloalkyl as described herein, for example, trifluoromethyl.

According to some of any of the embodiments of Formula IV as described herein, R₁₀ and R₁₁ are each a heteroalicyclic (e.g., are each morpholine); and R₃ is trifluoromethyl. In some of these embodiments, R₁, R₂, R₄ and R₅ are each hydrogen. In some of these embodiments, R₆ is hydrogen.

According to some of any of the embodiments of Formula IV as described herein, R₉ is hydrogen; and R₃ is trifluoromethyl. In some of these embodiments, R₁, R₂, R₄ and R₅ are each hydrogen. In some of these embodiments, R₆ is hydrogen.

According to some of any of the embodiments of Formula IV as described herein, R₁₀ and R₁₁ are each a heteroalicyclic (e.g., are each morpholine); and R₃ is trifluoromethyl. In some of these embodiments, R₁, R₂, R₄ and R₅ are each hydrogen. In some of these embodiments, R₆ is hydrogen.

According to some of any of the embodiments of Formula IV as described herein, R₉ is hydrogen; R₁₀ and R₁₁ are each a heteroalicyclic (e.g., are each morpholine); and R₃ is trifluoromethyl. In some of these embodiments, R₁, R₂, R₄ and R₅ are each hydrogen. In some of these embodiments, R₆ is hydrogen.

According to some of any of the embodiments of Formula IV as described herein, at least one of R₁-R₅ is hydroxy.

According to some of any of the embodiments of Formula IV as described herein, at least one of R₂, R₃ and R₄ is hydroxy.

According to some of any of the embodiments of Formula IV as described herein, at least two of R₂, R₃ and R₄ are each hydroxy.

According to some of any of the embodiments of Formula IV as described herein, R₂ and R₃ are each hydroxy, and R₁, R₄ and R₅ are each hydrogen. In some of these embodiments, R₆ is hydrogen.

According to some of any of the embodiments of Formula IV as described herein, R₂ and R₃ are each hydroxy, and R₁, R₄, R₅ and R₉ are each hydrogen. In some of these embodiments, R₆ is hydrogen.

According to some of any of the embodiments of Formula IV as described herein, R₁, R₄, R₅ and R₆ are each hydrogen. In some of these embodiments, R₉ is hydrogen.

According to some of any of the embodiments of Formula IV as described herein, R₁, R₄, R₅ and R₆ are each hydrogen; and R² and R₃ are each hydroxy. In some of these embodiments, R₉ is hydrogen.

According to some of any of the embodiments of Formula IV as described herein, R₁ R₄, R₅, R₆ and R₉ are each hydrogen; and R² and R₃ are each hydroxy.

Exemplary compounds of Formula IV include GLA5 and GLC26 (see, Table 2).

According to some of any of the embodiments described herein, the compound is (E)-4,4′-(6-(2-(4-(trifluoromethyl)benzylidene) hydrazinyl)-1,3,5-triazine-2,4-diyl)dimorpholine), which is also referred to herein as GLA5, as shown below. According to some of any of the embodiments described herein, the compound is GLA5 or a pharmaceutical acceptable salt thereof.

In some of any of the embodiments described herein, and the compound is (Z)-4,4′-(6-(2-(4-(trifluoromethyl)benzylidene) hydrazinyl)-1,3,5-triazine-2,4-diyl)dimorpholine, as shown below.

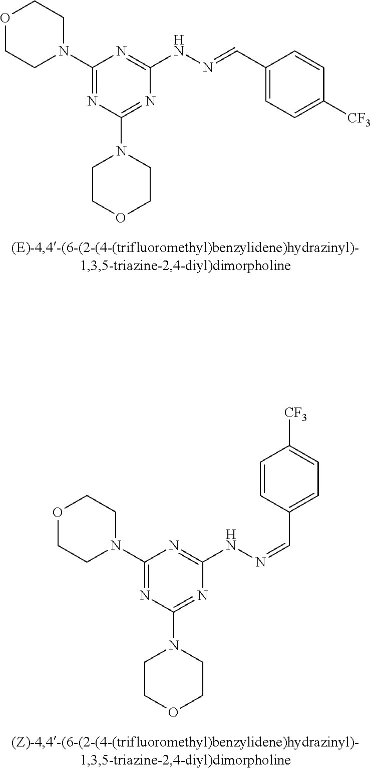 [SMILES: FC(F)(F)c1ccc(/C=N/Nc2nc(N3CCOCC3)nc(N3CCOCC3)n2)cc1 ; FC(F)(F)c1ccc(/C=N\Nc2nc(N3CCOCC3)nc(N3CCOCC3)n2)cc1]

According to some of any of the embodiments described herein, the compound is (E)-4-((2-(4,6-dimorpholino-1,3,5-triazin-2-yl)hydrazono)methyl)benzene-1,2-diol), which is also referred to herein as GLC26, as shown below. According to some of any of the embodiments described herein, the compound is GLC26 or a pharmaceutical acceptable salt thereof.

In some of any of the embodiments described herein, the compound is (Z)-4-((2-(4,6-dimorpholino-1,3,5-triazin-2-yl)hydrazono)methyl)benzene-1,2-diol, as shown below.

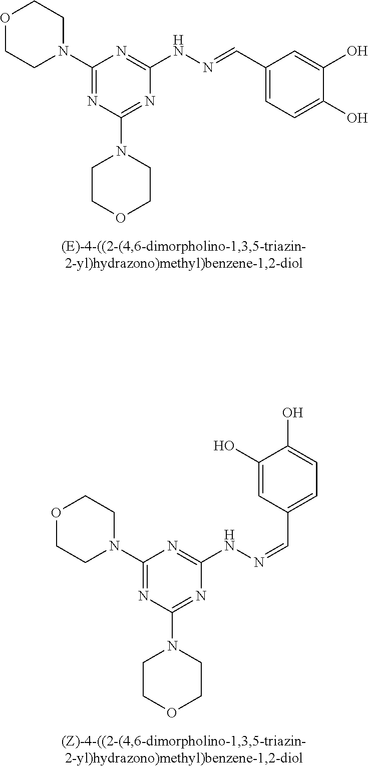 [SMILES: Oc1ccc(/C=N/Nc2nc(N3CCOCC3)nc(N3CCOCC3)n2)cc1O ; Oc1ccc(/C=N\Nc2nc(N3CCOCC3)nc(N3CCOCC3)n2)cc1O]

According to an aspect of some embodiments of the present invention there is provided a compound represented by Formula V:

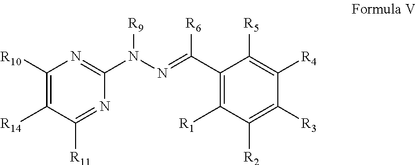

or a pharmaceutical acceptable salt thereof,
wherein:
R₁-R₆, R₁₀, R₁₁ and R₁₄ are each independently selected from the group consisting of hydrogen, alkyl, trihaloalkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, with the proviso that neither R₂ nor R₄ is hydroxy or alkoxy;
at least one of R₁-R₅ is a trihaloalkyl, as described herein in any of the respective embodiments of Formula IV and any combination thereof;
R₉ is alkyl, preferably a lower alkyl as described herein for Formula I; and
at least one of R₁₀ and R₁₁ is other than hydrogen.

According to some of any of the embodiments of Formula V as described herein, at least one or each of R₁₀ and R₁₁ is a substituted or non-substituted phenyl, as described herein in any of the respective embodiments of Formula I and any combination thereof.

According to some of any of the embodiments of Formula V as described herein, each of R₁₀ and R₁₁ is independently a substituted or non-substituted phenyl, as described herein in any of the respective embodiments of Formula I and any combination thereof, which can be the same or different.

According to some of any of the embodiments of Formula V as described herein, R₁₄ is hydrogen.

According to some of any of the embodiments of Formula V as described herein, at least one or each of R₁₀ and R₁₁ is a substituted or non-substituted phenyl, as described herein in any of the respective embodiments of Formula I and any combination thereof; and R₁₄ is hydrogen.

According to some of any of the embodiments of Formula V as described herein, each of R₁₀ and R₁₁ is independently a substituted or non-substituted phenyl, as described herein in any of the respective embodiments of Formula I and any combination thereof, which can be the same or different; and R₁₄ is hydrogen.

According to some of any of the embodiments of Formula V as described herein, when one or more of R₁-R₅ is a trihaloalkyl, the trihaloalkyl is a lower trihaloalkyl, of from 1 to 10, or from 1 to 8, or from 1 to 6, preferably of from 1 to 4, carbon atoms in length. The three halo atoms can be the same or different. In some embodiments the three halo atoms are the same and is some embodiments, the trihaloalkyl is a trifluoroalkyl, preferably a trifluoroalkyl of 1 to 4 carbon atoms in length. In exemplary embodiments, the trihaloalkyl is a trihalomethyl, preferably a trifluoromethyl.

According to some of any of the embodiments of Formula V as described herein, at least one of R₂, R₃ and R₄ is a trihaloalkyl (e.g., trihalomethyl, trifluoroalkyl or trifluoromethyl).

According to some of any of the embodiments of Formula V as described herein, R₁₀ and R₁₁ are each independently a substituted or non-substituted phenyl; and at least one of R₂, R₃ and R₄ is a trihaloalkyl, as defined herein.

According to some of any of the embodiments of Formula V as described herein, R₃ is a trihaloalkyl, as defined herein. In some of these embodiments, R₁, R₂, R₄ and R₅ are each independently selected from hydrogen, hydroxy, alkoxy and trifluoromethyl. In some of these embodiments, R₆ is hydrogen. In some of these embodiments, R₉ is a lower alkyl as described herein for Formula I.

According to some of any of the embodiments of Formula V as described herein, R₁₀ and R₁₁ are each independently a substituted or non-substituted phenyl; R₁₄ is hydrogen; and R₃ is a trihaloalkyl, as defined herein. In some of these embodiments, R₁, R₂, R₄ and R₅ are each independently selected from hydrogen, hydroxy, alkoxy and trifluoromethyl. In some of these embodiments, R₆ is hydrogen. In some of these embodiments, R₉ is a lower alkyl as described herein for Formula I.

According to some of any of the embodiments of Formula V as described herein, none of R₁-R₅ is hydroxy or alkoxy. In some of these embodiments, R₁-R₅ are each independently selected from hydrogen, alkyl, trihaloalkyl, alkenyl, alkynyl, cycloalkyl, aryl, heteroaryl, heteroalicyclic, and halo. In some of these embodiments, R₆ is hydrogen. In some of these embodiments, R₉ is a lower alkyl as described herein for Formula I. According to some of any of the embodiments of Formula V as described herein, R₁₀ and R₁₁ are each independently a substituted or non-substituted phenyl; and none of R₁-R₅ is hydroxy or alkoxy (e.g., R₁-R₅ are each independently selected from hydrogen, alkyl, trihaloalkyl, alkenyl, alkynyl, cycloalkyl, aryl, heteroaryl, heteroalicyclic, and halo, or any of the other substituents as defined herein). In some of these embodiments, R₆ is hydrogen. In some of these embodiments, R₉ is a lower alkyl as described herein for Formula I.

According to some of any of the embodiments of Formula V as described herein, R₁₀ and R₁₁ are each independently a substituted or non-substituted phenyl; R₃ is a trifluoromethyl; and none of R₁, R₂, R₄ and R₅ is hydroxy or alkoxy (e.g., R₁, R₂, R₄ and R₅ are each independently selected from hydrogen, alkyl, trihaloalkyl, alkenyl, alkynyl, cycloalkyl, aryl, heteroaryl, heteroalicyclic, and halo, or any of the other substituents as defined herein). In some of these embodiments, R₆ is hydrogen. In some of these embodiments, R₉ is a lower alkyl as described herein for Formula I.

According to some of any of the embodiments of Formula V as described herein, R₁, R₂, R₄ and R₅ are each hydrogen. In some of these embodiments, R₃ is hydrogen or a trihaloalkyl as defined herein. In some of these embodiments, R₆ is hydrogen. In some of these embodiments, R₉ is a lower alkyl as described herein for Formula I.

According to some of any of the embodiments of Formula V as described herein, R₁₀ and R₁₁ are each independently a substituted or non-substituted phenyl; and R₁, R₂, R₄, and R₅ are each hydrogen. In some of these embodiments, R₆ is hydrogen.

According to some of any of the embodiments of Formula V as described herein, R₁₀ and R₁₁ are each independently a substituted or non-substituted phenyl; R₃ is a trihaloalkyl (e.g., trifluoromethyl); and at least one, or at least two, or at least three, or each, of R₁, R₂ and R₄, and R₅ is hydrogen. In some of these embodiments, R₆ is hydrogen . . .

According to some of any of the embodiments of Formula V as described herein, R₉ is a linear, unsubstituted lower alkyl as described herein, for example, methyl.

According to some of any of the embodiments of Formula V as described herein, R₁₀ and R₁₁ are each independently a substituted or non-substituted phenyl; and R₉ is a linear, unsubstituted lower alkyl as described herein, for example, methyl.

According to some of any of the embodiments of Formula V as described herein, at least one of R₂, R₃ and R₄ is a trihaloalkyl as defined herein (e.g., trihalomethyl, trifluoromethyl); and R₉ is a linear, unsubstituted lower alkyl as described herein, for example, methyl.

According to some of any of the embodiments of Formula V as described herein, R₁₀ and R₁₁ are each independently a substituted or non-substituted phenyl; and at least one of R₂, R₃ and R₄ is a trihaloalkyl; and R₉ is a linear, unsubstituted lower alkyl as described herein, for example, methyl.

According to some of any of the embodiments of Formula V as described herein, R₃ is trifluoromethyl; and R₉ is a linear, unsubstituted lower alkyl as described herein, for example, methyl.

According to some of any of the embodiments of Formula V as described herein, none of R₁-R₅ is hydroxy or alkoxy, as described herein in any of the respective embodiments; and R₉ is a linear, unsubstituted lower alkyl as described herein, for example, methyl. In some of these embodiments, R₆ is hydrogen.

According to some of any of the embodiments of Formula V as described herein, R₁₀ and R₁₁ are each independently a substituted or non-substituted phenyl; and none of R₁-R₅ is hydroxy or alkoxy, as described herein in any of the respective embodiments; and R₉ is a linear, unsubstituted lower alkyl as described herein, for example, methyl. In some of these embodiments, R₆ is hydrogen.

According to some of any of the embodiments of Formula V as described herein, R₁₀ and R₁₁ are each independently a substituted or non-substituted phenyl; R₃ is a trihaloalkyl (e.g., trifluoromethyl); none of R₁, R₂, R₄- and R₅ is hydroxy or alkoxy, as described herein in any of the respective embodiments; and R₉ is a linear, unsubstituted lower alkyl as described herein, for example, methyl. In some of these embodiments, R₆ is hydrogen.

According to some of any of the embodiments of Formula V as described herein, R₁, R₂, R₄, R₅ and R₆ are each hydrogen; and R₉ is a linear, unsubstituted lower alkyl as described herein, for example, methyl. In some of these embodiments, R₃ is hydrogen, hydroxy, alkoxy or a trihaloalkyl, as defined herein. In some of these embodiments, R₆ is hydrogen.

According to some of any of the embodiments of Formula V as described herein, R₁₀ and R₁₁ are each independently a substituted or non-substituted phenyl; R₁, R₂, R₄, R₅ and R₆ are each hydrogen; and R₉ is a linear, unsubstituted lower alkyl as described herein, for example, methyl. In some of these embodiments, R₃ is hydrogen, hydroxy, alkoxy or a trihaloalkyl as defined herein.

According to some of any of the embodiments of Formula V as described herein, R₁₀ and R₁₁ are each independently a substituted or non-substituted phenyl; R₃ is trifluoromethyl; R₁, R₂, R₄, R₅ and R₆ are each hydrogen; and R₉ is a linear, unsubstituted lower alkyl as described herein, for example, methyl.

According to some of any of the embodiments of Formula V as described herein, R₉ is methyl.

According to some of any of the embodiments of Formula V as described herein, R₁₀ and R₁₁ are each independently a substituted or non-substituted phenyl; and R₉ is methyl.

According to some of any of the embodiments of Formula V as described herein, at least one of R₂, R₃ and R₄ is a trihaloalkyl; and R₉ is methyl.

According to some of any of the embodiments of Formula V as described herein, R₁₀ and R₁₁ are each independently a substituted or non-substituted phenyl; at least one of R₂, R₃ and R₄ is a trihaloalkyl; and R₉ is methyl.

According to some of any of the embodiments of Formula V as described herein, R₃ is trifluoromethyl; and R₉ is methyl.

According to some of any of the embodiments of Formula V as described herein, R₁₀ and R₁₁ are each independently a substituted or non-substituted phenyl; and R₃ is trifluoromethyl. In some of these embodiments, R₆ is hydrogen. In some of these embodiments, R₁, R₂, R₄ and R₅ are each independently selected from hydrogen, hydroxy, alkoxy and trihaloalkyl.

According to some of any of the embodiments of Formula V as described herein, none of R₁-R₅ is hydroxy or alkoxy, as described herein in any of the respective embodiments; and R₉ is methyl. In some of these embodiments, R₆ is hydrogen.

According to some of any of the embodiments of Formula V as described herein, R₁₀ and R₁₁ are each independently a substituted or non-substituted phenyl; none of R₁-R₅ is hydroxy or alkoxy, as described herein in any of the respective embodiments; and R₉ is methyl. In some of these embodiments, R₆ is hydrogen.

According to some of any of the embodiments of Formula V as described herein, R₁₀ and R₁₁ are each independently a substituted or non-substituted phenyl; R₃ is trifluoromethyl; and none of R₁-R₅ is hydroxy or alkoxy, as described herein in any of the respective embodiments; and R₉ is methyl. In some of these embodiments, In some of these embodiments, R₆ is hydrogen.

According to some of any of the embodiments of Formula V as described herein, R₁, R₂, R₄, R₅ and R₆ are each hydrogen; and R₉ is methyl. In some of these embodiments, R₃ is hydrogen, hydroxy, alkoxy or a trihaloalkyl such as trifluoromethyl.

According to some of any of the embodiments of Formula V as described herein, R₁₀ and R₁₁ are each independently a substituted or non-substituted phenyl; R₁, R₂, R₄, R₅ and R₆ are each hydrogen; and R₉ is methyl. In some of these embodiments, R₃ is hydrogen, hydroxy, alkoxy or a trihaloalkyl such as trifluoromethyl.

According to some of any of the embodiments of Formula V as described herein, R₁₀ and R₁₁ are each independently a substituted or non-substituted phenyl; R₃ is trifluoromethyl; and R₁, R₂, R₄, R₅ and R₆ are each hydrogen; and R₉ is methyl. According to some of any of the embodiments described the herein, compound is (E)-2-(1-methyl-2-(4-(trifluoromethyl)benzylidene) hydrazinyl)-4,6-diphenyl pyrimidine, which is also referred to herein as GLC19, as shown below. According to some of any of the embodiments described herein, the compound is GLC19 or a pharmaceutical acceptable salt thereof.

In some of any of the embodiments described herein, the compound is (Z)-2-(1-methyl-2-(4-(trifluoromethyl)benzylidene) hydrazinyl)-4,6-diphenylpyrimidine, as shown below.

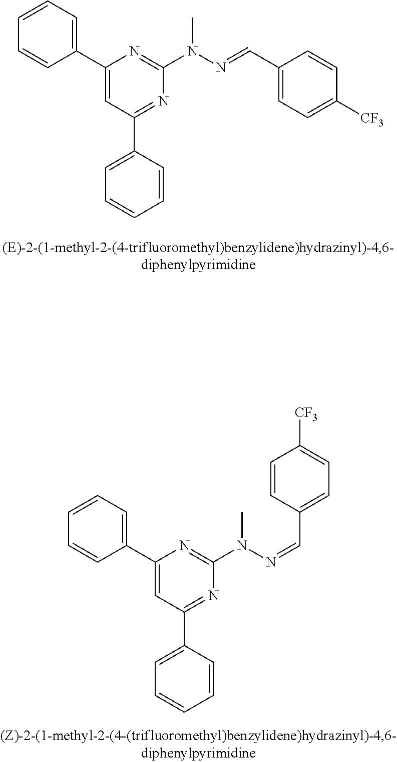 [SMILES: CN(/N=C/c1ccc(C(F)(F)F)cc1)c1nc(-c2ccccc2)cc(-c2ccccc2)n1 ; CN(/N=C\c1ccc(C(F)(F)F)cc1)c1nc(-c2ccccc2)cc(-c2ccccc2)n1]

Compounds according to any of the embodiments as described herein (e.g., of Formula I, II, III, IV or V), in which one or more of R₁-R₅ is OH, are presented herein as an “enol” tautomer, can undergo keto-enol tautomerization. Some embodiments of the present invention therefrom encompass also the “keto” tautomer of these compounds.

Exemplary keto-enol tautomers are presented in the following scheme for Compound GLB7.

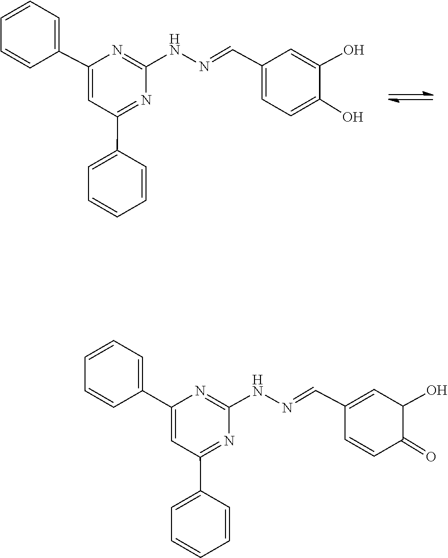 [SMILES: Oc1ccc(/C=N/Nc2nc(-c3ccccc3)cc(-c3ccccc3)n2)cc1O ; O=C1C=CC(/C=N/Nc2nc(-c3ccccc3)cc(-c3ccccc3)n2)=CC1O]

In some embodiments, compounds which present keto-enol tautomerization are in a form of the “enol” tautomer.

According to aspects of some embodiments of the present invention, there are provided processes of preparing the compounds as described herein in any of the respective embodiments. Exemplary such processes are described in further detail in the Examples section that follows.

In some of any of the embodiments described herein, compounds as described herein (e.g., having any one of Formulae I, III, IV and V) can be prepared by (a) nucleophilic aromatic substitution (S_{N}Ar) on an aryl bearing a leaving group (L) as described herein in the presence of a hydrazine (e.g., unsubstituted or mono-substituted hydrazine); and (b) Schiff-based reaction in the presence of an aryl aldehyde (e.g., substituted or unsubstituted), to provide a compound as described herein, as shown in the Examples section that follows.

In some of any of the embodiments described herein, compounds as described herein (e.g., having Formula II) can be prepared by (a) nucleophilic aromatic substitution (S_{N}Ar) on an aryl bearing a leaving group (L) as described herein in the presence of a hydrazine (e.g., unsubstituted or mono-substituted hydrazine); and (b) Schiff-based reaction in the presence of an aryl aldehyde (e.g., substituted or unsubstituted) and a reducing agent (e.g., sodium cyanoborohydride), to provide a compound as described herein, as shown in the Examples section that follows.

### Uses and Indications:

According to an aspect of some embodiments of the present invention there is provided a compound as described herein in any of the respective embodiments (e.g., of Formula I, II, III, IV or V) for use in activating PERK, or upregulating PERK activity.

According to an aspect of some embodiments of the present invention there is provided a compound as described herein in any of the respective embodiments (e.g., of Formula I, II, III, IV or V) for use in treating a medical condition (e.g., a disease or disorder) in which activating PERK, or upregulating PERK activity, is beneficial.

Herein and in the art, the term “PERK” refers to a protein also known as “PKR-like endoplasmic reticulum kinase” and “eIF2αK3” (eIF2α kinase 3).

Herein, the phrase “upregulating PERK activity” means increasing an activity of PERK and/or the processes and/or protein activities which result in the production of PERK. Upregulation may optionally be effected on the genomic and/or the transcript level by promoting transcription and/or translation of one or more proteins involved in the activity and/or production of PERK; and/or on the protein level by activating one or more proteins involved in increasing the activity of PERK (e.g., by stabilizing a protein to prevent its degradation, phosphorylation/dephosphorylation, increasing UPR as discussed herein, and the like).

An exemplary activity of PERK (which may be inhibited according to some embodiments described herein) is phosphorylation of eIF2α.

Activating PERK or upregulating PERK activity can therefore be determined, according to some embodiments of the present invention, by determining phosphorylation of eIF2α in the presence of a compound as described herein (optionally in comparison to the phosphorylation of eIF2α in the absence of the compound). Determining phosphorylation of eIF2α can be performed by methods known to those skilled in the art. Exemplary methods are provided in the Examples section that follows.

In some of any of the embodiments described herein, increasing or upregulating PERK activity is by at least 5%, or by at least 10%, or by at least 20%, or by at least 25%, or by at least 30%, or by at least 40%, or by at least 50%, or by at least 60 5, or by at least 70%, or by at least 80%, or by at least 90%, or by at least 100% (2-folds), or by more, for example, by 200% (3-folds), 300% (4-folds), 400% (5-folds) or even 1,000% (10-folds) or higher, relative to the PERK activity without a compound as defined herein. According to an aspect of some embodiments of the present invention, there is provided a use of a compound according to any one of the embodiments described herein in the manufacture of a medicament for treating a disease or disorder in which activation of PERK is beneficial.

According to an aspect of some embodiments of the present invention there is provided a method of activating PERK or upregulating PERK activity, or treating a medical condition (e.g., a disease or disorder) in which activation of PERK is beneficial, in a subject in need thereof, which comprises administering to the subject a therapeutically effective amount of a compound as described herein in any of the respective embodiments (e.g., of Formula I, II, III, IV or V).

Diseases in which such activation of PERK is beneficial (in the context of any of the respective embodiments described herein) include, for example, neurodegenerative diseases and disorders.

Examples of neurodegenerative diseases and disorders treatable by a compound described herein include, without limitation, Huntington's disease; Alzheimer's disease; Parkinson's disease; amyotrophic lateral sclerosis (ALS); prion disease (e.g., Creutzfeldt-Jakob disease, scrapie); Lewy-body dementia; spongiform encephalopathies; multiple sclerosis; glutamate neurotoxicity; motor neuron disease; restless legs syndrome (RLS) migraine; neurodegenerative disease or disorder associated with traumatic injury (e.g., concussion, blast injury and/or combat-related injury), bacterial and/or viral infection, ischemia and/or hypoxia (e.g., cerebral ischemia, ischemic/reperfusion injury in stroke, myocardial ischemia and/or renal ischemia); platelet aggregation, heart attack, cardiac hypertrophy, atherosclerosis and/or arteriosclerosis; spinal cord injury (e.g., partial or total spinal cord transection); frontotemporal dementias; AIDS-associated dementia; ataxias; and memory deficiencies (e.g., long-term memory impairment).

According to an aspect of some embodiments of the present invention there is provided a compound as described herein in any of the respective embodiments (e.g., of Formula I, II, III, IV or V), for use in treating a medical condition in which modulating (e.g., downregulating or upregulating) an unfolded protein response is beneficial.

According to an aspect of some embodiments of the present invention there is provided a compound as described herein in any of the respective embodiments (e.g., of Formula I, II, III, IV or V), for use in treating a medical condition in which upregulating an unfolded protein response is beneficial.

According to an aspect of some embodiments of the present invention there is provided a compound as described herein in any of the respective embodiments (e.g., of Formula I, II, III, IV or V), for use in the manufacture of a medicament for treating a medical condition in which modulating (e.g., upregulating) an unfolded protein response is beneficial.

According to an aspect of some embodiments of the present invention there is provided a compound as described herein in any of the respective embodiments (e.g., of Formula I, II, III, IV or V), for use in the manufacture of a medicament for treating a medical condition in which upregulating an unfolded protein response is beneficial.

According to an aspect of some embodiments of the present invention there is provided a method of modulating (e.g., upregulating) an unfolded protein response or of treating a medical condition in which modulating (e.g., upregulating) an unfolded protein response is beneficial, the method comprising administering to a subject in need thereof a therapeutically effective amount of a compound as described herein in any of the respective embodiments (e.g., of Formula I, II, III, IV or V).

Herein, the terms “unfolded protein response” and “UPR” refer to a cellular stress response associated with endoplasmic reticulum (ER) stress, which includes at least three components that counteract ER stress: stress gene expression, translational attenuation, and ER-associated protein degradation (ERAD). UPR typically includes activity by the transducer proteins XBP1, ATF6 and PERK. Therefore, compounds that activate any one of XBP1, ATF6 and PERK, are also capable of upregulating an unfolded protein response.

Herein, “upregulating” an unfolded protein response means increasing a degree of any (optionally all) of the processes and/or protein activities encompassed by an unfolded protein response, for example, by increasing an amount of a protein involved in the unfolded protein response and/or by activating the protein. Activating may optionally be effected on the genomic and/or the transcript level by promoting transcription and/or translation of one or more proteins involved in an unfolded protein response; and/or on the protein level by activating one or more proteins involved in an unfolded protein response (e.g., by phosphorylation/dephosphorylation, agonism, preventing cleavage of the protein, and the like).

Modulation of UPR may optionally be determined as an increase or a decrease in phosphorylation of eIF2a, for example, in the presence of a condition or compound (e.g., a compound as described herein in any of the respective embodiments) which induces integrated stress response (ISR) by, e.g., phosphorylation of translation initiation factor eIF2α (e.g., as exemplified herein).

Activation of UPR may optionally be determined as an increase in phosphorylation of eIF2α, for example, in the presence of a condition or compound (e.g., a compound as described herein in any of the respective embodiments) which induces integrated stress response (ISR) by, e.g., phosphorylation of translation initiation factor eIF2α (e.g., as exemplified herein).

Examples of diseases or disorders in which upregulating an unfolded protein response is beneficial include, without limitation, Alzheimer's disease, Parkinson's disease, Huntington's disease, Frontotemporal dementia and other tauopathies, ALS and prion disease (e.g., Creutzfeldt-Jakob disease).

In some embodiments, the compound according to any one of the embodiments described herein is for use in treating Huntington's disease.

According to an aspect of some embodiments of the present invention there is provided a compound as described herein in any of the respective embodiments (e.g., of Formula I, II, III, IV or V), for use in treating a medical condition (e.g., a disease or disorder) associated with an aggregation-prone protein.

According to an aspect of some embodiments of the present invention there is provided a compound as described herein in any of the respective embodiments (e.g., of Formula I, II, III, IV or V), for use in the manufacture of a medicament for treating a medical condition (e.g., a disease or disorder) associated with an aggregation-prone protein.

According to an aspect of some embodiments of the present invention there is provided a method of treating a medical condition (e.g., a disease or disorder) associated with an aggregation-prone protein, which comprises administering to a subject in need thereof a therapeutically effective amount of a compound as described herein in any of the respective embodiments (e.g., of Formula I, II, III, IV or V).

Examples of aggregation-prone proteins (also referred to herein and in the art as aggregation-forming proteins) include, but are not limited to, Z alpha1-antitrypsin, alpha-synuclein, tau, beta amyloid, SOD1, prion protein (prp), neuroserpin, islet amyloid protein (IAPP), ataxins (1-7), androgen receptor, atrophin 1, huntingtin and other polyglutamine repeat proteins, HOXD13 (synpolydactyly) and other polyalanine repeat proteins.

Examples of diseases or disorders associated with aggregation-prone proteins include, without limitation, Huntington's disease, amyloidosis, cataract, type II diabetes, cancer and memory deficiency (e.g., long-term memory impairment).

According to an aspect of some embodiments of the present invention, there is provided a use of a compound according to any one of the embodiments described herein in the manufacture of a medicament for treating a medical condition as described herein in any of the respective embodiments (e.g., Huntington's disease).

According to an aspect of some embodiments of the present invention, there is provided a method of treating a medical condition as described herein in any of the respective embodiments (e.g., Huntington's disease), the method comprising administering to a subject in need thereof a therapeutically effective amount of a compound according to any one of the embodiments described herein.

Further non-limiting examples (in addition to, e.g., Huntington's disease) of diseases or disorders that are treatable by compounds described herein (according to any of the respective embodiments described herein) include, without limitation, other neurodegenerative diseases and disorders such as, but not limited to, Alzheimer's disease, Parkinson's disease, amyotrophic lateral sclerosis (ALS), prion disease, Lewy-body dementia, spongiform encephalopathies, frontotemporal dementias, and ataxias; as well as other conditions involving protein aggregation such as, for example, amyloidosis, cataract, type II diabetes, cancer and memory deficiency (e.g., long term memory impairment).

In some embodiments, the medical condition is a viral infection.

According to an aspect of some embodiments of the present invention there is provided a compound as described herein in any of the respective embodiments (e.g., of Formula I, II, III, IV or V) for use in treating a viral infection.

According to an aspect of some embodiments of the present invention, there is provided a method of treating a viral infection in a subject in need thereof, the method comprising administering to the subject a compound as described herein in any of the respective embodiments (e.g., of Formula I, II, III, IV or V).

According to an aspect of some embodiments of the present invention, there is provided a method of reducing a population of a virus (e.g., in a subject in need thereof), the method comprising contacting the virus (e.g., in vivo, ex-vivo or in vitro) or a cell or tissue infected by the virus with a compound as described herein in any of the respective embodiments (e.g., of Formula I, II, III, IV or V).

According to an aspect of some embodiments of the present invention, there is provided a use of a PERK modulator or a compound capable of downregulating or upregulating PERK activity in treating a viral infection in a subject in need thereof and/or in the manufacturing of a medicament for treating a viral infection in a subject in need thereof and/or in reducing a population of a virus, as described herein.

According to an aspect of some embodiments of the present invention, there is provided a use of a PERK activator or a compound capable of upregulating PERK activity in treating a viral infection in a subject in need thereof and/or in the manufacturing of a medicament for treating a viral infection in a subject in need thereof and/or in reducing a population of a virus, as described herein.

Herein, the phrase “treating a viral infection” means reducing and/or alleviating symptoms of a viral infection, inhibiting growth and/or killing the virus, reducing a population of the virus, and the like. Treating a viral infection further refers to the amelioration of any biological or pathological endpoints that is mediated in part by the presence of the virus in the subject, and whose outcome can be affected by reducing the level of viral gene products present.

According to some embodiments, treating a viral infection comprises reducing a load of virus in the subject.

By “reducing a load” it is meant reducing a population of a virus that causes the viral infection, by, for example, killing and/or inhibiting growth of the virus.

As used herein, the term “subject” includes mammals, preferably human beings at any age that suffer from the pathology. Preferably, this term encompasses individuals who are at risk to develop the pathology.

A viral infection according to any of the embodiments described herein may be associated with any virus species and/or strain.

Herein, the term “virus” refers to an agent that replicates only inside living cells of an organism, and encompasses agents composed solely of a nucleic acid, such as viroids. Examples of viruses include, without limitation, double strand DNA viruses, such as adenoviruses, herpesviruses (e.g., varicella zoster virus, herpes simplex virus-1 and/or herpes simplex virus-2), polyomaviruses (e.g., JC virus), and poxviruses; single strand DNA viruses, such as parvoviruses; double strand RNA viruses, such as reoviruses (e.g., epizootic hemorrhagic disease virus); (+)-single strand RNA viruses, such as coronaviruses (e.g., coronavirus HKU1, coronavirus NL63, coronavirus 229E, coronavirus OC43, Middle East respiratory syndrome coronavirus (MERS-CoV) and/or SARS-COV), flaviviruses (e.g., hepatitis C virus and/or West Nile virus), hepeviruses (e.g., hepatitis E virus), picornaviruses (e.g., hepatitis A virus, enteroviruses and/or rhinoviruses, such as human enteroviruses and/or rhinoviruses) and togaviruses; (−)-single strand RNA viruses, such as orthomyxoviruses (e.g., influenza A virus and/or influenza B virus), filoviruses (e.g., Ebola virus), paramyxoviruses (e.g., parainfluenza virus type 1, 2, 3 and/or 4), pneumoviruses (e.g., respiratory syncytial virus and/or human metapneumovirus) and rhabdoviruses (e.g., vesicular stomatitis Indiana virus); RNA retroviruses; DNA retroviruses, such as hepadnaviruses (e.g., hepatitis B virus); satellite viruses, such as deltaviruses (e.g., hepatitis D virus); and viroids.

According to some of any of the embodiments described herein, the viral infection is associated with double strand DNA viruses, single strand DNA viruses, double strand RNA viruses, (+)-single strand RNA viruses, (−)-single strand RNA viruses, RNA retroviruses; DNA retroviruses, satellite viruses, and/or viroids.

Exemplary viruses that cause disease include, but are not limited to, those set forth in Table A hereinbelow.

TABLE A

| | Baltimore | | |
|---|---|---|---|
| Family | group | Important species | Envelopment |
| Adenoviridae | Group I | Adenovirus | non-enveloped |
| | (dsDNA) | | |
| Herpesviridae | Group I | Herpes simplex, type 1, Herpes | enveloped |
| | (dsDNA) | simplex, type 2, Varicella-zoster virus, | |
| | | Epstein-Barr virus, Human | |
| | | cytomegalovirus, Human herpesvirus, | |
| | | type 8 | |
| Papillomaviridae | Group I | Human papillomavirus | non-enveloped |
| | (dsDNA) | | |
| Polyomaviridae | Group I | BK virus, JC virus | non-enveloped |
| | (dsDNA) | | |
| Pox viridae | Group I | Smallpox | enveloped |
| | (dsDNA) | | |
| Hepadnaviridae | Group VII | Hepatitis B virus | enveloped |
| | (dsDNA-RT) | | |
| Parvoviridae | Group II | Parvovirus B19 | non-enveloped |
| | (SSDNA) | | |
| Astroviridae | Group IV | Human astrovirus | non-enveloped |
| | (positive-sense | | |
| | SSRNA) | | |
| Caliciviridae | Group IV | Norwalk virus | non-enveloped |
| | (positive-sense | | |
| | SSRNA) | | |
| Picornaviridae | Group IV | coxsackievirus, hepatitis A virus, | non-enveloped |
| | (positive-sense | poliovirus, rhinovirus | |
| | SSRNA) | | |
| Coronaviridae | Group IV | Severe acute respiratory syndrome | enveloped |
| | (positive-sense | virus | |
| | SSRNA) | | |
| Flaviviridae | Group IV | Hepatitis C virus, yellow fever virus, | enveloped |
| | (positive-sense | dengue virus, West Nile virus, TBE | |
| | ssRNA) | virus | |
| Family | Baltimore | Important species | Envelopment |
| | group | | |
| Togaviridae | Group IV | Rubella virus | enveloped |
| | (positive-sense | | |
| | SSRNA) | | |
| Hepeviridae | Group IV | Hepatitis E virus | non-enveloped |
| | (positive-sense | | |
| | SSRNA) | | |
| Retroviridae | Group VI | Human immunodeficiency virus (HIV) | enveloped |
| | (ssRNA-RT) | | |
| Orthomyxoviridae | Group V | Influenza virus | enveloped |
| | (negative- | | |
| | sense ssRNA) | | |
| Arenaviridae | Group V | Lassa virus | enveloped |
| | (negative- | | |
| | sense ssRNA) | | |
| Bunyaviridae | Group V | Crimean-Congo hemorrhagic fever | enveloped |
| | (negative- | virus, Hantaan virus | |
| | sense ssRNA) | | |
| Filoviridae | Group V | Ebola virus, Marburg virus | enveloped |
| | (negative- | | |
| | sense ssRNA) | | |
| Paramyxoviridae | Group V | Measles virus, Mumps virus, | enveloped |
| | (negative- | Parainfluenza virus, Respiratory | |
| | sense ssRNA) | syncytial virus, | |
| Rhabdoviridae | Group V | Rabies virus | enveloped |
| | (negative- | | |
| | sense ssRNA) | | |
| Unassigned | Group V | Hepatitis D | enveloped |
| | (negative- | | |
| | sense ssRNA) | | |
| Reoviridae | Group III | Rotavirus, Orbivirus, Coltivirus, Banna | non-enveloped |
| | (dsRNA) | virus | |

According to some of any of the embodiments described herein, the virus is an RNA virus.

According to some of any of the embodiments described herein, the virus is coronavirus, rhabdovirus and/or reovirus.

According to specific embodiments, the virus is a coronavirus.

According to specific embodiments, a clinical manifestation of Coronavirus infection includes symptoms selected from the group consisting of inflammation in the lung, alveolar damage, fever, cough, shortness of breath, diarrhea, organ failure, pneumonia and/or septic shock.

As used herein, “Coronavirus” refers to enveloped positive-stranded RNA viruses that belong to the family Coronaviridae and the order Nidovirales.

The coronavirus according to any of the respective embodiments described herein is optionally a betacoronavirus, for example, an embecovirus (a.k.a. lineage A), sarbecovirus (a.k.a. lineage B), merbecovirus (a.k.a. lineage C), nobecovirus (a.k.a. lineage D), and hibecovirus. Exemplary betacoronaviruses include SARS-related coronavirus (a species of sarbecovirus), human coronavirus OC43, and human coronavirus HKU1, including any strains thereof (e.g., SARS-COV-2).

Alternatively or additionally, examples of coronaviruses which are contemplated herein include, but are not limited to, 229E, NL63, OC43, and HKU1 with the first two classified as antigenic group 1 and the latter two belonging to group 2, typically leading to an upper respiratory tract infection manifested by common cold symptoms.

However, Coronaviruses, which are zoonotic in origin, can evolve into a strain that can infect human beings leading to fatal illness. Thus particular examples of Coronaviruses contemplated herein are SARS-COV, Middle East respiratory syndrome Coronavirus (MERS-CoV), and the recently identified SAR-COV-2 [causing 2019-nCOV (also referred to as “COVID-19”)].

It would be appreciated that any Coronavirus strain is contemplated herein even though SAR-COV-2 is emphasized in a detailed manner.

According to specific embodiments, the virus is a Rhabdovirus.

A clinical manifestation of Rhabdovirus infection includes symptoms selected from the group consisting of fever, headache, muscle weakness, malaise, nausea, vomiting, diarrhea, abdominal pain, photophobia, confusion, seizures, and paralysis.

As used herein, “Rhabdovirus” refers to enveloped negative-stranded RNA viruses that belong to the family Rhabdoviridae and the order Mononegavirales.

The Rhabdovirus according to any of the respective embodiments described herein is optionally a member of the genus Lyssavirus or Vesiculovirus, for example, rabies lyssavirus or Vesicular stomatitis Indiana virus (VSV). Additional Rhabdoviruses include Chandipura virus and Mokola virus, including any strains thereof.

It would be appreciated that any Rhabdovirus strain is contemplated herein even though VSV is emphasized in a detailed manner.

According to some of any of the embodiments described herein, the virus is a Severe acute respiratory syndrome coronavirus (SARS-COV) or a Vesicular stomatitis Indiana virus (VSV).

In some embodiments, the viral infection treatable using a compound as described herein in any of the respective embodiments is associated with a virus other than a coronavirus or a vesicular stomatitis Indiana virus (VSV). Examples of other viruses include, but are not limited to, CMV (cytomegalovirus), HRV (human rhinoviruses), hepatovirus A, HMV (human meningo virus), and HIV (human immunodeficiency virus).

According to some of any of the embodiments described herein, the treatment results in inhibition of exit of a viral glycoprotein from endoplasmic reticulum.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

As used herein throughout, the term “trihaloalkyl” refers any alkyl as defined herein, that is substituted by three halo substituents, as defined herein. The halo substituents can be on any carbon of the alkyl, optionally on one of the carbons of the alkyl, for example, on a terminal carbon atom of the alkyl. The three halo substituents can be the same or different, preferably the same.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

Herein, the terms “amine” and “amino” each refer to either a —NR′R″ group or a —N⁺R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R′″ are not (for example) carbonyl, C-carboxy or amide, as these groups are defined herein.

An “azide” group refers to a —N═N⁺═N⁻ group.

An “alkoxy” group refers to any of an —O-alkyl, —O-alkenyl, —O-alkynyl, —O-cycloalkyl, and —O-heteroalicyclic group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and −S-heteroalicyclic group, as defined herein.

A “thioaryloxy” group refers to both an —S-aryl and an —S-heteroaryl group, as defined herein.

A “carbonyl” or “acyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O)OH group.

An “oxo” group refers to a ═O group.

An “imine” group refers to a ═N—R′ group, where R′ is as defined herein.

An “oxime” group refers to a ═N—OH group.

A “hydrazone” group refers to a ═N—NR′R″ group, where each of R′ and R″ is as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R'S(═O)₂—NR″— group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S) NR″— group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a-PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R′″ are as defined herein.

A “guanidinyl” group refers to an —RaNC(═NRd)-NRbRc group, where each of Ra, Rb, Rc and Rd can be as defined herein for R′ and R″.

A “guanyl” or “guanine” group refers to an RaRbNC(═NRd)- group, where Ra, Rb and Rd are as defined herein.

For any of the embodiments described herein, the compound described herein may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any isomer, stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an(S) configuration and any combination, and compounds according to some embodiments of the present invention, can have any their chiral centers exhibit an (R) or an(S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

### Pharmaceutical Composition:

The compound of some embodiments of the invention can be administered to an organism per se, or in a pharmaceutical composition which further comprises a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compound accountable for the biological effect.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, nonirritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include crosslinked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredient (e.g., a compound according to any of the respective embodiments described herein) effective to prevent, alleviate or ameliorate symptoms of a disorder (e.g., a viral infection, or a disease or disorder associated with a viral infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels (e.g., blood levels) of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In any of the methods and uses as described herein, a compound according to the present invention can be used in combination with an additional therapeutically active agent that is usable in treating an indicated medical condition and/or is usable as capable of modulating (e.g., activating; upregulating) PERK activity. The additional active agent can be co-administered or co-formulated or otherwise used in combination with a compound according to the present embodiments.

As used herein, the term “subject” includes mammals, preferably human beings at any age which suffer from the pathology. Preferably, this term encompasses individuals who are at risk to develop the pathology.

It is expected that during the life of a patent maturing from this application many relevant medical conditions in which activating PERK is beneficial will be uncovered and the scope of the term “medical condition” is intended to include all such new technologies a priori.

As used herein the term “about” refers to +10% or +5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

Materials: Tunicamycin (Tun) and other common reagents were obtained from Merck.

MK-28 was synthesized according to procedures described in WO 2017/216792.

Microwave irradiation experiments: Microwave irradiation experiment was performed using CEM Discover® SP machine, using the setup parameters as presented in Table 1.

TABLE 1

| | |
|---|---|
| Setup parameters 1 | Setup parameters 2 |

| | | |
|---|---|---|
| Method type | Dynamic | Dynamic |

| | | | | |
|---|---|---|---|---|
| Pressure limit | 250 | PSI | 250 | PSI |
| Vessel Type | 10 | ml | 10 | ml |
| Temperature | 100° | C. | 115° | C. |
| Power | 100 | W | 100 | W |

| | | |
|---|---|---|
| Hold time (h:m:s) | 00:08:00 | 01:00:00 |
| PreMix | No | No |
| Stirring | High | High |
| Cooling | On | On |

LC-MS: Waters Autopurification system analytical module equipped with SQD2 MS detector at the following conditions: a. LC: Waters XBridge BEH300 C4 (3.5 μm, 4.6 mm×100 mm) using a 8-minute gradient from 95:5 Water: acetonitrile (both with 0.1% formic acid) to acetonitrile; b. MS: scan mode 100-1000.

¹H and ¹³C NMR: ¹H and ¹³C NMR spectra were measured on Bruker 400 (400 MHZ ¹H, 100 MHz ¹³C). Chemical shifts values (δ) are reported in ppm (calibration of spectra to the residual peak of TMS: δ=0.0 ppm(s) for ¹H NMR; δ=0.0 ppm for ¹³C NMR if not mentioned otherwise). All the proton spectra reported as following: δ value (multiplicity, J coupling constant (in Hz), number of nuclei). Multiplicity contractions used: (s)-singlet, (d)-doublet, (dd)-doublet of doublet, (t)-triplet, (q)-quartet, (m)-multiplet, and (br)-broad signal.

Cell culture and transfections: Mouse embryonic fibroblasts (MEF) cells were grown as described in the art. Murine striatal cell line expressing full-length polyQ-expanded Htt (STHdhQ111/111) cells were grown as described in Leitman et al. [PLOS One 2014, 9 (3): e90803]. HEK 293 and VERO cells were grown in DMEM supplemented with 10% bovine calf serum at 37° C. under 5% CO₂.

Apoptosis: Apoptosis was measured by cell cycle progression using propidium iodide (PI), considering the fraction beneath G0/G1.

Statistical analysis: Generally, the results are expressed as mean±standard deviation of 3 independent experiments. Apoptosis in the presence of tunicamycin and in the absence of other compounds was normalized to 100 to allow comparison between experiments. Significance by Two-tailed Student's t-test.

### Example 1

### Chemical Syntheses

Exemplary compounds according to some of the present embodiments, e.g., GLA2, GLA5, GLB11, GLC19, GLC21, GLC22 and GLC26, were synthesized generally according to procedures such as described in WO 2017/216792, adopted to the desirable compounds. Chemical structures were confirmed by ¹H and ¹³C NMR, mass spectrometry (MS), and HPLC (not shown).

An exemplary two-step general synthetic pathway for Compounds of Formulae I and III-V in which R₆═H is depicted in Scheme 1 below and is described in further detail thereafter.

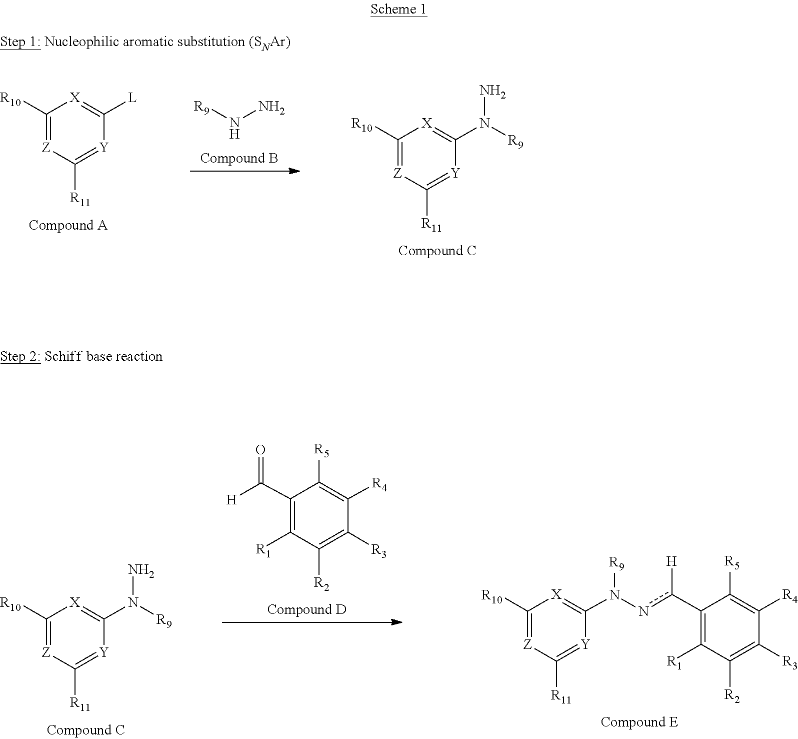

wherein, for Scheme 1:
X is N or CR₁₂;
Y is N or CR₁₃;
Z is N or CR₁₄; and
L is a leaving group (as are well-known in the art and as described herein),
and all other variables are defined herein for the respective formulae.

As used herein, the phrase “leaving group” describes a labile atom, group or chemical moiety that readily undergoes detachment from an organic molecule during a chemical reaction, while the detachment is typically facilitated by the relative stability of the leaving atom, group or moiety thereupon. Typically, any group that is the conjugate base of a strong acid can act as a leaving group. Representative examples of suitable leaving groups according to some of the present embodiments include, without limitation, trichloroacetimidate, acetate, tosylate, triflate, sulfonate, azide, halide, hydroxy, thiohydroxy, alkoxy, cyanate, thiocyanate, nitro and cyano.

### Step 1:

Into a vial equipped with a stirring bar, a substituted aryl or heteroaryl (Compound A in Scheme 1, 1.0 equivalent) and hydrazine (R₉—NH—NH₂, Compound B in Scheme 1, 20 equivalents) are added at room temperature.

The vial is fitted with a snap-on cap, the solution stirs for 10 seconds, and placed in a CEM Discover® SP microwave with setup parameters 2 as described in the Materials and Experimental Methods section hereinabove. The reaction mixture is thereafter cooled in the refrigerator overnight, and the resultant precipitate is filtered, washed with cold 1:1 EtOH/water solution and dried by lyophilization.

### Step 2:

Into a vial equipped with a stirring bar, the hydrazino-diphenylpyrimidine obtained in Step 1 (Compound C is Scheme 1; 1.0 equivalent), a solution of an aldehyde (Compound D in Scheme 1; 1.5 equivalents) in a polar protic solvent such as isopropanol (0.22 M), and a catalytic amount of acetic acid are added at room temperature. The vial is fitted with a snap-on cap, the solution is stirred for 10 seconds, and placed in a CEM Discover® SP microwave with setup parameters 1 as described in the Materials and Experimental Methods section hereinabove. The reaction mixture is thereafter cooled in the refrigerator overnight, and the resultant precipitate is filtered, washed with cold isopropanol and dried by lyophilization.

In an exemplary synthesis, the compound GLB7 was prepared as follows:

### Preparation of 2-hydrazineyl-4,6-diphenylpyrimidine (GLB7-Im-1)

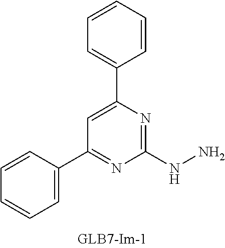 [SMILES: NNc1nc(-c2ccccc2)cc(-c2ccccc2)n1]

GLB7-Im-1 was prepared using the general Nucleophilic aromatic substitution described in Step 1 hereinabove, starting from 2-chloro-4,6-diphenylpyrimidine as compound A (0.6 mmol, 1.0 equivalent) and hydrazine monohydrate as compound B (20 mmol, 33 equivalents), to provide GLB7-Im-1 as compound C (119 mg, 75% yield) in at least 98% HPLC purity at diode array.

HPLC: RT 5.38 minutes, using an 8-minute gradient from 95:5 Water: acetonitrile (both with 0.1% formic acid) to acetonitrile.
MS: ESI+263.40 [M+H].

¹H NMR (400 MHZ, DMSO) δ 8.29 (m, 4H), 7.76 (s, ¹H), 7.59-7.48 (m, 6H), 4.37 (s, 2H).

### Preparation of 4-((2-(4,6-diphenylpyrimidin-2-yl)hydrazineylidene) methyl)benzene-1,2-diol (GLB7)

GLB7 was prepared using the general Schiff base reaction as described in Step 2 hereinabove, starting from GLB7-Im-1 as compound C (0.44 mmol, 1.0 equivalent), and 3,4-dihydroxybenzaldehyde (0.66 mmol, 1.5 equivalents) as compound D, in 2 ml isopropanol, to provide GLB7 (168 mg, 96% yield) as a Z: E mixture of about 1:1, in at least 98% HPLC purity at diode array.

HPLC: RT 6.09 minutes, using an 8-minute gradient from 95:5 Water: acetonitrile (both with 0.1% formic acid) to acetonitrile.

MS: ES+383.48 [M+H].

NMR: ¹H NMR (400 MHZ, DMSO) δ 11.09 (s, ¹H), 9.24 (s, ¹H), 9.19 (s, ¹H), 8.37-8.29 (m, 4H), 8.07 (s, ¹H), 7.93 (s, ¹H), 7.58 (m, 6H), 7.27 (d, J=1.8 Hz, ¹H), 6.92 (dd, J=8.2, 1.8 Hz, ¹H), 6.79 (dd, J=8.1, 1.5 Hz, ¹H). ¹³C NMR (101 MHz, DMSO) δ 165.40, 160.94, 147.36, 146.03, 142.77, 131.15, 129.13, 127.57, 127.25, 119.88, 112.85, 104.23.

In an exemplary synthesis, the compound GLB11 was prepared as follows:

### Preparation of 2-hydrazineyl-4,6-diphenylpyrimidine (GLB7-Im-2)

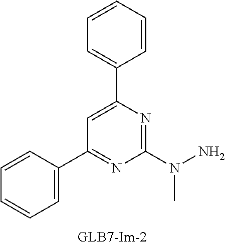 [SMILES: CN(N)c1nc(-c2ccccc2)cc(-c2ccccc2)n1]

GLB7-Im-2 was prepared using the general Nucleophilic aromatic substitution described in Step 1 hereinabove, starting from 2-chloro-4,6-diphenylpyrimidine as compound A and methylhydrazine as compound B, to provide GLB7-Im-2 as compound C.

### Preparation of 4-((2-(4,6-diphenylpyrimidin-2-yl)-2-methylhydrazinyl)methyl)benzene-1,2-diol (GLB11)

GLB11 was prepared using the general Schiff base reaction as described in Step 2 hereinabove, starting from dissolving GLB7-Im-2 as compound C (1.0 equivalent) and NaBH₃CN (1.0 equivalents) in 8 ml MeOH, and 3,4-dihydroxybenzaldehyde (1.2 equivalents) was then added as compound D and stirred overnight at room temperature, to provide GLB11.

R_{f} (DCM: few drops of MeOH)=0.35.

HPLC: RT 5.81 minutes, using a 10-minute gradient from 1:1 water: acetonitrile to acetonitrile.

The structure is further confirmed by NMR and MS.

In an exemplary synthesis, the compound GLC21 was prepared as follows:

Preparation of 5-((2-(pyrimidin-2-yl)hydrazono)methyl)benzene-1,2,3-triol (GLC21):

GLC21 was prepared using the general Schiff base reaction as described in Step 2 hereinabove, starting from 2-hydrazinylpyrimidine as compound C (1.0 equivalent) and 3,4,5-trihydroxybenzaldehyde (1.5 equivalents) as compound D to provide GLC21, in at least 98% HPLC purity at diode array.

HPLC: RT 1.54 minutes, using a 10-minute gradient from 1:1 water: acetonitrile to acetonitrile.

MS: 247.4 [M+H].

The structure is further confirmed by NMR.

In an exemplary synthesis, the compound GLA2 was prepared as follows:

### Preparation of 4-((2-methyl-2-(pyrimidin-2-yl)hydrazono)methyl)benzene-1,2-diol (GLA2)

GLA2 was prepared using the general Schiff base reaction as described in Step 2 hereinabove, starting from 2-(1-methylhydrazinyl) pyrimidine as compound C and 3,4-dihydroxybenzaldehyde as compound D to provide GLA2.

The structure is further confirmed by NMR, HPLC and MS.

In an exemplary synthesis, the compound GLC22 was prepared as follows:

### Preparation of 5-((2-(pyrimidin-2-yl)hydrazono)methyl)benzene-1,2,3-triol (GLC22)

GLC22 was prepared using the general Schiff base reaction as described in Step 2 hereinabove, starting from 2-(1-methylhydrazinyl) pyrimidine as compound C and 3,4,5-trihydroxybenzaldehyde as compound D to provide GLC22, in at least 98% HPLC purity at diode array.

HPLC: RT 5.92 minutes, using a 10-minute gradient from 1:1 water: acetonitrile to acetonitrile.

MS: 261.4 [M+H].

The structure is further confirmed by NMR.

In an exemplary synthesis, the compound GLA5 was prepared as follows:

### Preparation of 4,4′-(6-(2-(4-(trifluoromethyl)benzylidene) hydrazinyl)-1,3,5-triazine-2,4-diyl)dimorpholine (GLA5)

GLA5 was prepared using the general Schiff base reaction as described in Step 2 hereinabove, starting from 4,4′-(6-hydrazinyl-1,3,5-triazine-2,4-diyl)dimorpholine as compound C and 4-(trifluoromethyl)benzaldehyde as compound D to provide GLA5 as a yellow solid.

The structure is further confirmed by NMR, HPLC and MS.

In an exemplary synthesis, the compound GLC19 was prepared as follows:

### Preparation of 4,4′-(6-(2-(4-(trifluoromethyl)benzylidene) hydrazinyl)-1,3,5-triazine-2,4-diyl)dimorpholine (GLC19)

GLC19 was prepared using the general Schiff base reaction as described in Step 2 hereinabove, starting from GLB7-Im-2 as compound C (1.0 equivalents), 4-(trifluoromethyl)benzaldehyde as compound D (1.5 equivalents) and acetic acid (0.3 equivalents) to provide GLC19.

HPLC: RT 6.98 minutes, using a 10-minute gradient from 1:1 water: acetonitrile to acetonitrile.

MS: 433.5 [M+H].

The structure is further confirmed by NMR.

Table 2 below presents the chemical structures and chromatographic purities (as determined by HPLC at diode array) of exemplary compounds prepared using the above-described synthetic protocols.

TABLE 2

| Compound | Purity |
|---|---|
| 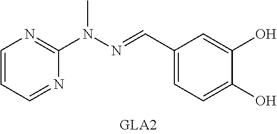 [SMILES: CN(/N=C/c1ccc(O)c(O)c1)c1ncccn1] | >98% |
| 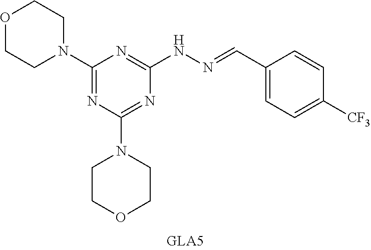 [SMILES: FC(F)(F)c1ccc(/C=N/Nc2nc(N3CCOCC3)nc(N3CCOCC3)n2)cc1] | >98% |
| 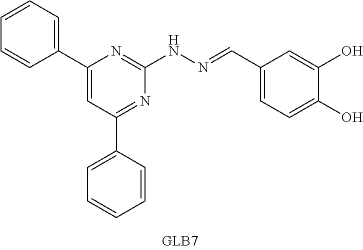 [SMILES: Oc1ccc(/C=N/Nc2nc(-c3ccccc3)cc(-c3ccccc3)n2)cc1O] | >98% |
| 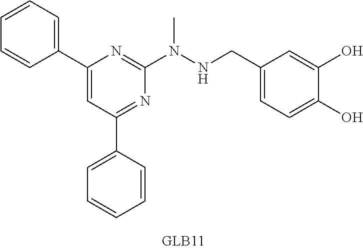 [SMILES: CN(NCc1ccc(O)c(O)c1)c1nc(-c2ccccc2)cc(-c2ccccc2)n1] | >97% |
| 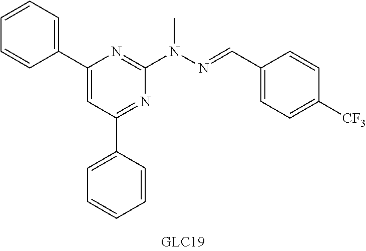 [SMILES: CN(/N=C/c1ccc(C(F)(F)F)cc1)c1nc(-c2ccccc2)cc(-c2ccccc2)n1] | >98% |
| 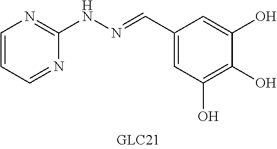 [SMILES: Oc1cc(/C=N/Nc2ncccn2)cc(O)c1O] | >98% |
| 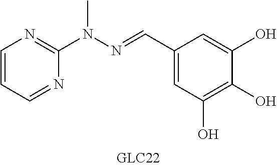 [SMILES: CN(/N=C/c1cc(O)c(O)c(O)c1)c1ncccn1] | >98% |
| 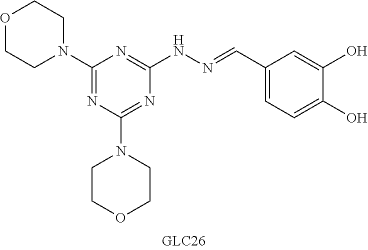 [SMILES: Oc1ccc(/C=N/Nc2nc(N3CCOCC3)nc(N3CCOCC3)n2)cc1O] | >95% |

### Example 2

### Effect of Exemplary Compounds on ER Stress-Induced Apoptosis

Previous studies by some of the present inventors have demonstrated that PERK activators such as MK-28 rescue murine striatal cell line expressing full-length polyQ-expanded Htt (STHdh^{Q111/111} cells) from UPR-induced apoptosis [Ganz et al. (2020) supra]. In order to assess the effect of the exemplary compounds on apoptosis, similar assays were performed in mouse embryonic fibroblasts (MEF; FIG. 1) and in STHdhQ111/111 cells (FIG. 2).

As shown in FIG. 1, the exemplary compounds GLB7, GLB11 and GLC22 reduced apoptosis by from about 40% to about 80% (at 10 μM) in mouse embryonic fibroblasts, in a dose-dependent manner.

As shown in FIG. 2, in STHdh^{Q111/111} cells, GLB11 and GLC19 reduced apoptosis by about 20% apoptosis (at 10 μM).

Overall, these data indicate that in MEF cells, cytotoxicity was generally reduced by the tested compounds, indicating that these compounds express activity as PERK activators.

### Example 3

### Solubility and Lipophilicity

The solubility and lipophilicity of GLB7 and GLB11, two exemplary compounds according to some embodiments of the present invention, were measured compared to the PERK activator MK-28.

The results are presented in Table 3. As can be seen, the exemplary compounds GLB7 and GLB11 showed improved solubility compared to MK-28 (>10-fold and >4-fold higher solubilities, respectively). A decreased lipophilicity was observed for GLB7, which is typically a favorable characteristic when pursuing the design of novel drugs.

TABLE 3

| Compound | Solubility (μM) | Lipophilicity (logD_{7.4}) |
|---|---|---|
| MK-28 | 1.4 | 4.5 |
| GLB7 | 16 | 3.6 |
| GLB11 | 6 | 4.8 |

### Example 4

### Effect of Exemplary Compounds on PERK Activation

An exemplary compound according to some embodiments of the present invention, GLB7, was tested in comparison with MK-28 for its ability to activate PERK.

For this purpose, HEK293 cells were untreated or treated with 5, 10 or 20 μM GLB7 or MK-28. After 24 hours, the cells were lysed and the phosphorylated eIF2α levels were measured relatively to the total eIF2α (a substrate of PERK).

As can be seen in FIGS. 3A-B, treatment with 5 μM GLB7 caused about a 70% increase in eIF2α phosphorylation relative to total eIF2α, thus indicating PERK activation by the exemplary compound GLB7. An increase in eIF2α phosphorylation relative to total eIF2α of 50% with 10 μM GLB7, 65% with 20 μM GLB7 and 30% to 50% with 5-20 μM MK-28 was also observed.

These results indicate that at treatment using the lowest examined dose of 5 μM, treatment with GLB7 resulted in higher levels of PERK activation.

### Example 5

### Effect of Exemplary Compounds on Viral Activity

The ability of the exemplary compounds to affect viral activity is determined by the following experiments:
(a) assessing the effect of the exemplary PERK activators on viral replication via in-vitro treatment of uninfected and viral-infected (e.g., SARS-COV-2 infected, vesicular stomatitis Indiana virus (VSV) infected) cells (e.g., Vero E6 cells, HEK293 cells), optionally at different multiplicities of infection (MOI) (e.g., 0.1, 1), and measuring of viral titer using methods known in the art at different time intervals (e.g., 16 hours, 24 hours) in comparison with data from untreated cells as control.

An antiviral activity in the presence of the exemplary compounds is indicated by a decrease in viral titer in comparison with untreated infected cells.
(b) investigating ER retention by the exemplary compounds in the presence of viral-infected (e.g., VSV infected) cells (e.g., HEK293 cells) optionally at different multiplicities of infection (MOI) (e.g., 0.1, 1), and quantifying (using, e.g., Western blot) viral biomarkers (e.g., maturation degree of N-linked oligosaccharides which is indicated by, e.g., the ratio between Endo H-sensitive (ER-localized) and Endo H-resistant (Golgi/post-Golgi-localized) forms of the glycoprotein in lysed cells 24 hours post-infection) in comparison with its degree in untreated control cells.

ER retention in the presence of the exemplary compounds is indicated, e.g., by a decrease in the degree of maturation (e.g., decrease in Endo H-resistance).
(c) the specificity of the exemplary compounds is tested, e.g., by measuring the effect of exemplary compounds on oligosaccharide maturation of cellular proteins (e.g., H2b, ACE2) and viral proteins (e.g., viral replication proteins, e.g., Endo H-resistance) in transfected (e.g., with VSV) cells (e.g., HEK293) in comparison with untreated transfected cells.

Specificity of the exemplary compounds is indicated by inhibition of the viral proteins while having minimal to no effect on cellular proteins.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-50. (canceled)

51. A compound represented by Formula II: 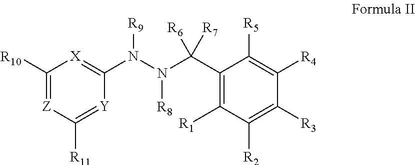
or a pharmaceutical acceptable salt thereof,
wherein:
X is N or CR₁₂;
Y is N or CR₁₃;
Z is N or CR₁₄;
R₁-R₇ and R₁₀-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino;
and
R₈ and R₉ are each independently hydrogen or alkyl,
provided that:
R₁₀ and R₁₁ are each independently a substituted or non-substituted phenyl; and/or
at least two of R₂, R₃ and R₄ is hydroxy.

52. The compound of claim 51, wherein:
X and Y are each N and Z is CR₁₄.

53. The compound of claim 51, wherein at least one of R₈ and R₉ is alkyl.

54. The compound of claim 51, wherein R₂ and R₃ are each hydroxy.

55. A compound represented by Formula I: 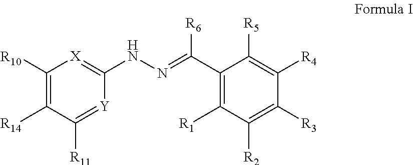
or a pharmaceutical acceptable salt thereof,
wherein:
X is N or CR₁₂;
Y is N or CR₁₃; and
R₁-R₆ and R₁₀-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino,
provided that:
at least two of R₂, R₃ and R₄ are each hydroxy; and/or
R₁₀ and R₁₁ are each independently a substituted or non-substituted phenyl.

56. The compound of claim 55, wherein at least one, or each, of X and Y is N.

57. The compound of claim 55, wherein R₂, R₃ and R₄ are each hydroxy.

58. A compound selected from:
a compound represented by Formula III: 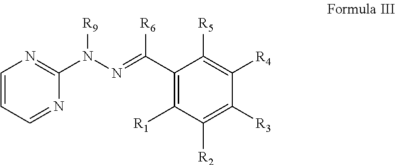
or a pharmaceutical acceptable salt thereof,
wherein:
R₁-R₆ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino; and
R₉ is alkyl,
a compound represented by Formula IV: 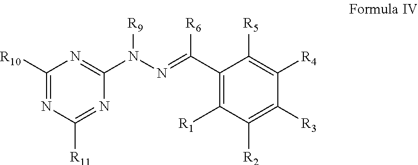
or a pharmaceutical acceptable salt thereof,
wherein:
R₁-R₆, R₁₀ and R₁₁ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino;
R₉ is hydrogen or alkyl; and
at least one, or each, of R₁₀ and R₁₁ is a heteroalicyclic,
and
a compound represented by Formula V: 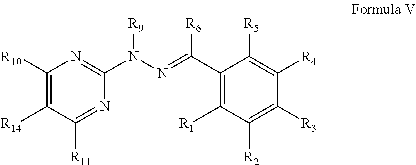
or a pharmaceutical acceptable salt thereof,
wherein:
R₁-R₆, R₁₀, R₁₁ and R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, with the proviso that neither R₂ nor R₄ is hydroxy or alkoxy;
at least one of R₂-R₄ is a trihaloalkyl;
R₉ is alkyl; and
at least one of R₁₀ and R₁₁ is other than hydrogen.

59. The compound of claim 58, represented by Formula III, wherein at least two of R₁-R₅ are each independently hydroxy or alkoxy.

60. The compound of claim 58, represented by Formula III, wherein at least two, or each, of R₂, R₃ and R₄ are each hydroxy.

61. The compound of claim 58, represented by Formula IV, wherein at least one of R₁-R₅, or at at least one of R₂-R₄, is a trihaloalkyl.

62. The compound of claim 58, represented by Formula IV, wherein at least one of R₁-R₅ is hydroxy and/or at least two of R₂, R₃ and R₄ are each hydroxy.

63. The compound of claim 58, represented by Formula III, wherein at least two of R₂, R₃ and R₄ are each hydroxy.

64. The compound of claim 58, represented by Formula V, wherein wherein R₁₀ and R₁₁ are each independently a substituted or non-substituted phenyl.

65. A method of treating a medical condition selected from a medical condition in which activating PERK is beneficial; a medical condition associated with an aggregation-prone protein and a medical condition in which upregulating an unfolded protein response is beneficial, the method comprising administering to a subject in need thereof a therapeutically effective amount of the compound of claim 51.

66. The method of claim 65, wherein said medical condition is selected from Huntington's disease, amyloidosis, cataract, type II diabetes, cancer, a viral infection, and memory deficiency.

67. A method of treating a medical condition selected from a medical condition in which activating PERK is beneficial; a medical condition associated with an aggregation-prone protein and a medical condition in which upregulating an unfolded protein response is beneficial, the method comprising administering to a subject in need thereof a therapeutically effective amount of the compound of claim 55.

68. The method of claim 67, wherein said medical condition is selected from Huntington's disease, amyloidosis, cataract, type II diabetes, cancer, a viral infection, and memory deficiency.

69. A method of treating a medical condition selected from a medical condition in which activating PERK is beneficial; a medical condition associated with an aggregation-prone protein and a medical condition in which upregulating an unfolded protein response is beneficial, the method comprising administering to a subject in need thereof a therapeutically effective amount of the compound of claim 58.

70. The method of claim 69, wherein said medical condition is selected from Huntington's disease, amyloidosis, cataract, type II diabetes, cancer, a viral infection, and memory deficiency.
